(19) United States
(12) Patent Application Publication
(10) Pub. No.: US 2004/0033972 A1
(43) Pub. Date: Feb. 19, 2004

(54) Treatment of mycobacterium tuberculosis with antisense polynucleotides

(76) Inventors: Marcus A. Horwitz, Los Angeles, CA (US); Gunter Harth, Los Angeles, CA (US); Paul C. Zamecnik, Boston, MA (US)

Correspondence Address: GATES & COOPER LLP, HOWARD HUGHES CENTER, 6701 CENTER DRIVE WEST, SUITE 1050, LOS ANGELES, CA, 90045, US

(21) Appl. No.: 10/168,244

(22) PCT Filed: Dec. 20, 2000
(86) PCT No.: PCT/US00/34688

(51) Int. Cl. A61K 48/00; A61K 31/496; A61K 31/4745; A61K 31/455

(52) U.S. Cl. 514/44; 536/23.2; 514/291; 514/354; 514/15; 514/252.13

(57) ABSTRACT

Methods of inhibiting the proliferation of Mycobacterium tuberculosis comprising contacting Mycobacterium tuberculosis with an effective amount of a polynucleotide complementary to an mRNA transcript expressed by Mycobacterium tuberculosis are provided. Typical methods of the invention utilize phosphorothioate modified antisense polynucleotides (PS-ODNs) against the mRNA of M.tuberculosis genes such as glutamine synthetase, aroA,ask, groES, and the genes of the Antigen 85 complex.

This application claims the benefit of U.S. provisional patent application No. 60/171,929, filed Dec. 22, 1999, the entire contents of which are incorporated herein by reference.

This invention was made with Government support under Grant Nos. AI 31338 and AI 42925, awarded by the National Institutes of Health. The Government has certain tights in this invention.

### FIELD OF THE INVENTION

The present invention relates to the use of antisense polynucleotides as prophylactic and therapeutic agents in the treatment of Mycobacterium tuberculosis infection.

### BACKGROUND OF THE INVENTION

Tuberculosis (TB) has been a major health problem for most of recorded history and Mycobacterium tuberculosis remains one of the world's most significant pathogens. Responsible for millions of new cases of tuberculosis annually (see e.g. Pablo-Mendez et al., (1998) New Engl. J. Med. 338, 1641-1649), it is the leading cause of death from a single infectious agent. While the incidence of the disease declined in parallel with advancing standards of living since at least the mid-nineteenth century, in spite of the efforts of numerous health organizations worldwide, the eradication of tuberculosis has never been achieved, nor is imminent

TB is acquired by the respiratory route; actively infected individuals spread this infection efficiently by coughing or sneezing “droplet nuclei” which contain viable bacilli. Overcrowded living conditions and shared air spaces are especially conducive to the spread of TB, underlying the increase in instances that have been observed in the U.S. in prison inmates and among the homeless in larger cities.

Alarmingly, outbreaks of TB cases resistant to at least two of the most effective anti-TB drugs (rifampin and isoniazid) are being reported in hospitals and correctional facilities with evidence of transmission to human immunodeficiency virus (HIV) negative individuals. Approximately half the patients with acquired immune deficiency syndrome (AIDS) will acquire a mycobacterial infection, with TB being an especially devastating complication. AIDS patients are at higher risks of developing clinical TB and anti-TB treatment seems to be less effective. Consequently, the infection often progresses to a fatal disseminated disease.

Presently an extremely disturbing phenomenon is this emergence of drug resistant M. tuberculosis, with the rate of new TB cases proven resistant to at least one standard drug increased from 10 percent in the early 1980s to 23 percent in 1991. Consequently, there is an immediate need for novel therapeutic compounds that will inhibit or halt the replication and infection of cells by Mycobacterium tuberculosis. The emergence of multidrug-resistant M. tuberculosis underscores the need for new approaches to combat this pathogen (see e.g. Pablo-Mendez et al., (1998) New Engl. J. Med. 338, 1641-1649). The compounds and methods for modulating the expression of Mycobacterium tuberculosis proteins as described herein meet that need.

### SUMMARY OF THE INVENTION

The invention disclosed herein comprises compounds and methods for treating or preventing Mycobacterium tuberculosis infection (tuberculosis) using antisense compounds such as antisense polynucleotides.

In a typical embodiment, the invention comprises a method for treating or preventing Mycobacterium tuberculosis infection using antisense or other site-specific polynucleotides directed against the mRNA or DNA of the gene encoding M. tuberculosis glutamine synthetase. An illustrative embodiment consists of a method of inhibiting Mycobacterium tuberculosis glutamine synthetase protein expression comprising contacting a Mycobacterium tuberculosis bacterium with an effective amount of an antisense compound comprising an antisense polynucleotide that hybridizes to a Mycobacterium tuberculosis glutamine synthetase polynucleotide, wherein the antisense polynucleotide hybridizes to a region of the Mycobacterium tuberculosis glutamine synthetase polynucleotide encoding the glutamine synthetase protein, thereby inhibiting Mycobacterium tuberculosis glutamine synthetase protein expression. In a specific embodiment of this method, the polynucleotide is selected from the group consisting of 5′-GAC GTC GTC GGG CGT CTT-3′ (SEQ ID NO: 18), 5′-CAT GCC GGA CCC GTT GTC GCC-3′ (SEQ ID NO: 19) and 5′-CCA CAG CGA CTG ATG ACA GTG CAT-3′ (SEQ ID NO: 20).

In another typical embodiment, the invention comprises a method for treating or preventing Mycobacterium tuberculosis infection using antisense or other site-specific polynucleotides directed against the mRNA or DNA of the M. tuberculosis aroA gene. In another typical embodiment, the invention comprises a method for treating or preventing Mycobacterium tuberculosis infection using antisense or other site-specific polynucleotides directed against the mRNA or DNA of the M. tuberculosis ask gene. In yet another typical embodiment, the invention comprises a method for treating or preventing Mycobacterium tuberculosis infection using antisense or other site-specific polynucleotides directed against the mRNA or DNA of genes encoding the M. tuberculosis 30/32 kDa (Antigen 85) extracellular protein complex.

In preferred methods of the invention, the antisense polynucleotide is relatively devoid of secondary structures. In typical methods of the invention, the polynucleotide is a phosphorothioate modified antisense polynucleotide. In a variation of the preferred methods of the invention, the method comprises contacting the Mycobacterium tuberculosis with both a polynucleotide as well as an effective amount of an antibiotic capable of inhibiting the proliferation of Mycobacterium tuberculosis such as rifampin, isoniazid, amikacin, ethambutol and polymyxin B nonapeptide. In yet another variation of the preferred methods, multiple polynucleotides targeting different regions of one or more genes and/or gene transcripts are used to treat or prevent Mycobacterium tuberculosis infection.

Yet another embodiment of the method consists of a process for producing an antisense compound that inhibits the expression of a Mycobacterium tuberculosis gene (e.g. glutamine synthetase) by synthesizing a antisense polynucleotide of about 15 to about 50 nucleobases in length capable of hybridizing to a portion of polynucleotide encoding a Mycobacterium tuberculosis glutamine synthetase protein and then comparing the levels of Mycobacterium tuberculosis glutamine synthetase protein expression in a Mycobacterium tuberculosis culture exposed to an effective amount of the antisense polynucleotide levels in a control culture. A related embodiment consists of an antisense polynucleotide produced according to this process.

As a detailed illustrative embodiment of the invention, we disclose the effects on M. tuberculosis of phosphorothioate modified antisense polynucleotides (PS-ODNs) against the mRNA of glutamine synthetase, an enzyme whose export is associated with pathogenicity and with the formation of a poly-L-glutamate/glutamine cell wall structure. Treatment of virulent M. tuberculosis with 10 μM antisense PS-ODNs reduced glutamine synthetase activity and expression by 25-50% depending upon whether one, two, or three different PS-ODNs were used and the PS-ODN's specific target site on the mRNA. Treatment with PS-ODNs of a recombinant strain of Mycobacterium smegmatis expressing M. tuberculosis glutamine synthetase selectively inhibited the recombinant enzyme but not the endogenous enzyme for which the mRNA transcript was mismatched by 2-4 nucleotides. Treatment of M. tuberculosis with the antisense PS-ODNs also reduced the amount of poly-L-glutamate/glutamine in the cell wall by 24%. Finally, treatment with antisense PS-ODNs reduced M. tuberculosis growth by 0.7 logs (1 PS-ODN) to 1.25 logs (3 PS-ODNs) but had no effect on the growth of M. smegmatis, which does not export glutamine synthetase nor possess the poly-L-glutamate/glutamine cell wall structure. The experiments indicate that the antisense PS-ODNs enter the cytoplasm of M. tuberculosis and bind to their cognate targets.

The disclosure provided herein demonstrates the feasibility of using antisense ODNs in the antibiotic armamentarium against M. tuberculosis.

### BRIEF DESCRIPTION OF THE DRAWINGS

FIGS. 1A-1B show the inhibition of M. tuberculosis Erdman glutamine synthetase activity by antisense PS-ODNs. FIG. 1A shows the inhibition of cellular glutamine synthetase activity of M. tuberculosis Erdman by antisense PS-ODNs. FIG. 1B shows the inhibition of extracellular glutamine synthetase activity of M. tuberculosis Erdman by antisense PS-ODNs. Duplicate M. tuberculosis Erdman cultures were grown for two weeks in 1-2 ml 7H9 medium alone and thereafter in the absence or the presence of antisense PS-ODNs, individually or combined, at concentrations of 10 μM. At each time point indicated, cultures were harvested and analyzed for glutamine synthetase activity by the transfer assay as described (see e.g. Harth et al., (1994) Proc. Natl. Acad. Sci. USA 91, 9342-9346). All data points had a standard deviation of ≦10%, even when multiple PS-ODNs were used.

FIGS. 2A-2D show the inhibition of glutamine synthetase activity in M. smegmatis 1-2c wildtype and its recombinant isotype expressing the M. tuberculosis glutamine synthetase. FIG. 2A shows the inhibition of cellular glutamine synthetase activity of M. smegmatis 1-2c wildtype by antisense PS-ODNs. FIG. 2B shows the inhibition of extracellular glutamine synthetase activity of M. smegmatis 1-2c wildtype by antisense PS-ODNs. FIG. 2C shows the inhibition of cellular glutamine synthetase activity of M. smegmatis 1-2c+rM.tb. GS by antisense PS-ODNs. FIG. 2D shows the inhibition of extracellular glutamine synthetase activity of M. smegmatis 1-2c+rM.tb. GS by antisense PS-ODNs. Duplicate M. smegmatis cultures were grown for two days in 1-2 ml 7H9 medium alone and thereafter in the absence or the presence of antisense PS-ODNs, individually or combined, at concentrations of 10 μM. At each time point indicated, cultures were harvested and analyzed for glutamine synthetase activity by the transfer assay as described Harth et al., (1994) Proc. Natl. Acad. Sci. USA 91, 9342-9346). Note that the cellular enzyme activity of the recombinant strain is a mixture of two enzyme activities—the endogenous glutamine synthetase (60%) and recombinant M. tuberculosis glutamine synthetase (40%). In contrast, the extracellular enzyme activity of the recombinant strain is almost exclusively M. tuberculosis glutamine synthetase (≧99%). All data points had a standard deviation of <15%.

FIGS. 3A-3B show the inhibition of cell proliferation of M. tuberculosis Erdman and M. smegmatis 1-2c wildtype and recombinant strain by antisense PS-ODNs. FIG. 3A shows the inhibition of cell proliferation of M. tuberculosis Erdman broth cultures by antisense PS-ODNs. FIG. 3B shows the inhibition of cell proliferation of M. smegmatis 1-2c±rM.tb. GS broth cultures by antisense PS-ODNs. Duplicate bacterial cultures were grown for six weeks (M. tuberculosis) or six days (M. smegmatis) in 1-2 ml 7H9 medium in the presence of the antisense PS-ODNs, individually or combined, at concentrations of 10 μM. At each time point, cultures were harvested, washed, serially diluted, and plated on 7H11 agar medium. Viable bacteria were enumerated after an incubation period of 2 weeks (M. tuberculosis) or 3 days (M. smegmatis). Standard deviations varied between 15-20%. There were no significant differences in growth of M. smegmatis 1-2c in the presence or absence of PS-ODNs.

FIGS. 4A-4C show the inhibition of cell proliferation of M. tuberculosis Erdman by antisense PS-ODNs in the presence of various concentrations of ethambutol or polymyxin B nonapeptide. FIG. 4A shows the inhibition of cell proliferation of M. tuberculosis Erdman broth cultures by antisense PS-ODNs in the presence of various concentrations of ethambutol (EMB). FIG. 4B shows the inhibition of cell proliferation of M. tuberculosis Erdman broth cultures by antisense PS-ODNs in the presence of various concentrations of polymyxin B nonapeptide (PMBN). FIG. 4C shows the inhibition of cell proliferation of M. tuberculosis Erdman broth cultures by antisense PS-ODNs in the presence of various concentrations of ethambutol (EMB) and polymyxin B nonapeptide (PMBN). Duplicate bacterial cultures were grown for six weeks in 1-2 ml 7H9 medium in the presence of the antisense PS-ODNs, individually or combined, at concentrations of 10 μM and 0, 0.1, 0.25, or 0.5 μg/ml ethambutol (EMB) and/or polymyxin B nonapeptide (PMBN) to “soften” the cell wall. At each time point, cultures were harvested, washed, serially diluted, and plated on 7H11 agar medium. Viable bacteria were enumerated after an incubation period of 2 weeks (M. tuberculosis) or 3 days (M. smegmatis). Standard deviations varied between 15-20%. Values for single PS-ODNs were combined to yield one dashed line; the range of values for single PS-ODNs is indicated by the broad, solid vertical bar.

FIG. 5 shows a graph of Val 23 with and without Ethambutol (ETH) (5 μg/ml).

FIG. 6 shows a graph of Val 24 with and without Ethambutol (ETH) (5 μg/ml).

FIG. 7 shows a graph of Val 25 with and without Ethambutol (ETH) (5 μg/ml).

FIG. 8 shows a graph of Val 26 with and without Ethambutol (ETH) (5 μg/ml).

FIG. 9 shows a graph of Val 33 with and without Ethambutol (ETH) (5 μg/ml).

FIG. 10 shows a graph of Val 34 with and without Ethambutol (ETH) (5 μg/ml).

FIG. 11 shows a graph of Val 41 with and without Ethambutol (ETH) (5 μg/ml).

FIG. 12 shows a graph of ML10 with and without Ethambutol (ETH) (5 μg/ml) and added once vs. weekly.

FIG. 13 shows the inhibition of cell proliferation of M. tuberculosis Erdman broth cultures by modified antisense PS-ODN 269-275 at 10 μM. This Figure shows that 269-275-DAO was more effective at 10 μM than PS-ODN 269-275 at inhibiting the growth of M. tuberculosis. The control ODN yielded no inhibition of growth, i.e. it was equivalent to no ODN being added.

FIG. 14 shows the inhibition of cell proliferation of M. smegmatis 1-2c±rM.tb. GS broth cultures by modified antisense PS-ODN 269-275 at 10 μM. This Figure shows that 269-275-DAO does not inhibit M. smegmatis.

FIG. 15 shows the inhibition of cell proliferation of M. tuberculosis Erdman broth cultures by modified antisense PS-ODN 269-275 at 10 μM. This Figure shows that only 5N-269-275 PS-ODN was more effective than PS-ODN 269-275 and that there was a trend toward greater inhibition with higher “N” groups. (5N>4N>3N/2N/1N).

FIGS. 16A-16E show the modified ODNs (derivatives with 5′, 3′ and 5′-3′ amino linkers) as described and evaluated in Example 9.

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Ausubel et al., Current Protocols in Molecular Biology, Wiley Interscience Publishers, (1995). As appropriate, procedures involving the use of commercially available kits and reagents are generally carried out in accordance with manufacturer defined protocols and/or parameters unless otherwise noted. In addition, a variety of art accepted definitions and methods for manipulating, evaluating and utilizing antisense compounds are well known in the art and are widely used as a standard practice in the field of biotechnology. Such common terms and practices are provided, for example in U.S. Pat. No. 6,140,126, which is incorporated herein by reference and which recites a variety of the common terms and methodologies illustrated below.

As discussed in detail below, the present invention employs oligomeric antisense compounds, particularly polynucleotides for use in modulating the expression and function of nucleic acid molecules encoding M. tuberculosis genes such as glnA, aroA, ask, groES, and the 30/32 kDa extracellular protein complex [30, 32A, 32B extracellular proteins (Antigens 85B, 85A, and 85C respectively)] by modulating the amount of protein produced from these M. tuberculosis genes. In descriptions of the invention provided herein, embodiments of a single gene ate used (for example the glutamine synthetase gene) to illustrate typical embodiments of the invention that apply to all of the methods and compositions for modulating glutamine synthetase, aroA, ask, groES and the 30/32 kDa extracellular protein complex genes. In this context, artisans understand that discussing typical embodiments directed to a single species (i.e. glutamine synthetase) when the embodiments are commonly applicable to the other species disclosed herein (e.g.. aroA, Ask, GroES or one of the Antigen 85 proteins) eliminates unnecessary redundancy in the descriptions of the invention.

Modulating the function of M. tuberculosis genes such as glutamine synthetase is accomplished by providing antisense compounds which specifically hybridize with one or mote nucleic acids encoding glutamine synthetase. As used herein, the terms “target nucleic acid” and “polynucleotide encoding glutamine synthetase” encompass DNA encoding glutamine synthetase, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of a M. tuberculosis gene such as glutamine synthetase. In the context of the present invention, “modulation” means either an increase (stimulation) or a decrease (inhibition) in the expression of a gene. In the context of the present invention, inhibition is the preferred form of modulation of gene expression and mRNA is a preferred target.

It is preferred to target specific nucleic adds in employing antisense technology. “Targeting” an antisense compound to a particular nucleic acid, in the context of this invention, is a multistep process. The process usually begins with the identification of a nucleic add sequence whose function is to be modulated. In the present invention, the target is a nucleic acid molecule encoding M. tuberculosis proteins such as glutamine synthetase. The targeting process also includes determination of a site or sites within this gene for the antisense interaction to occur such that the desired effect, e.g., detection or modulation of expression of the protein, will result. Within the context of the present invention, a preferred site is the region within the open reading frame (ORF) of the gene. Skilled artisans will understand that regions outside of the open reading frame (ORF) of the gene can also be targeted and that antisense compounds that target such regions and function to hybridize to M. tuberculosis polynucleotides and consequently inhibit protein expression can also be produced by the processes described herein.

The ORF of the various Mycobacterium tuberculosis genes discussed herein is known in the art (see e.g. Cole et al, (1998) Nature 393, 537-544). Moreover, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG or 5′-UUG,and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or N-formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding glutamine synthetase, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terns “stop codon region” and “translation termination codon region” refer to a portion of such an mRNA or gene that encompasses from about 25 to about 50 contiguous nucleotides in either direction (i.e., 5′ or 3′) from a translation termination codon.

Once one or more target sites have been identified, polynucleotides are chosen which are sufficiently complementary to the target, i.e., hybridize sufficiently well and with sufficient specificity, to give the desired effect. In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise base pairing between two nucleotides. For example, if a nucleotide at a certain position of a polynucleotide is capable of hydrogen bonding with a nucleotide at the same position of a DNA or RNA molecule, then the polynucleotide and the DNA or RNA ate considered to be complementary to each other at that position. The polynucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementary or precise pairing such that stable and specific binding occurs between the polynucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementary to avoid non-specific binding of the antisense compound to non-target sequences under conditions in which specific binding is desired, i.e., under physiological conditions in the case of in vivo assays or therapeutic treatment, or in the case of in vitro assays, under conditions in which the assays are performed.

Antisense compounds are commonly used as research reagents and diagnostics. For example, antisense polynucleotides, which are able to inhibit gene expression with exquisite specificity, are often used by those of ordinary skill to elucidate the function of particular genes. In the context of pathogenic organisms that have developed resistance to an existing antibiotic armamentarium (such as Mycobacterium tuberculosis), an elucidation of the function of particular genes (e.g. glutamine synthetase) is crucial for the development of the next generation of antibiotic therapeutics. Antisense modulation of protein activity has therefore been harnessed for research involving the treatment of disease. Antisense compounds are also used in other contexts, for example to distinguish between functions of various members of a biological pathway. Moreover, antisense compounds such as those disclosed herein can be used in a number of in vitro contexts, for example to prevent the growth of bacteria in mediums practitioners wish to keep free of contamination.

The specificity and sensitivity of antisense is also harnessed by those of skill in the art for therapeutic uses. Antisense polynucleotides have been employed as therapeutic moieties in the treatment of disease states in animals and man. Antisense polynucleotides have been safely and effectively administered to humans and numerous clinical trials are presently underway. It is thus established that polynucleotides can be useful therapeutic modalities that can be configured to be useful in treatment regimes for treatment of cells, tissues and animals, especially humans (see e.g. Barker et al., (1996) Proc. Natl. Acad. Sci. USA 93, 514-518, Nakaar et al., (1999) J. Biol. Chem. 274, 5083-5087 and Lisziewicz et al., (1992) Proc. Natl. Acad. Sci. USA 89, 11209-11213).

In the context of this invention, the term “polynucleotide” refers to an oligomer or polymer of ribonucleic acid (RNA) or deoxyribonucleic acid DNA) or mimetics thereof This term includes polynucleotides composed of naturally-occurring nucleobases, sugars and covalent internucleoside (backbone) linkages as well as polynucleotides having non-naturally-occurring portions which function similarly. Such modified or substituted polynucleotides are often preferred over native forms because of desirable properties such as, for example, enhanced cellular uptake, enhanced affinity for nucleic acid target and increased stability in the presence of nucleases.

While antisense polynucleotides are a preferred form of antisense compound, the present invention comprehends other oligomeric antisense compounds, including but not limited to polynucleotide mimetics such as are described below. The antisense compounds in accordance with this invention preferably comprise from about 8 to about 30 nucleobases. Particularly preferred are antisense polynucleotides comprising from about 8 to about 30 nucleobases (i.e. from about 8 to about 30 linked nucleosides). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming polynucleotides, the phosphate groups covalently link adjacent nucleosides to one another to form a linear polymeric compound. In turn the respective ends of this linear polymeric structure can be further joined to form a circular structure, however, open linear structures are generally preferred. Within the polynucleotide structure, the phosphate groups are commonly referred to as forming the internucleoside backbone of the polynucleotide. The normal linkage or backbone of RNA and DNA is a 3′ to 5′ phosphodiester linkage.

Specific examples of preferred antisense compounds useful in this invention include polynucleotides containing modified backbones or non-natural internucleoside linkages. As defined in this specification, polynucleotides having modified backbones include those that retain a phosphorus atom in the backbone and those that do not have a phosphorus atom in the backbone. For the purposes of this specification, and as sometimes referenced in the art, modified polynucleotides that do not have a phosphorus atom in their internucleoside backbone can also be considered to be oligonucleosides.

Preferred modified polynucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but ate not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Preferred modified polynucleotide backbones that do not include a phosphorus atom therein have backbones that ate formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other preferred polynucleotide mimetics, both the sugar and the internucleoside linkage, i.e., the backbone, of the nucleotide units ate replaced with novel groups. The base units are maintained for hybridization with an appropriate nucleic acid target compound. One such oligomeric compound, a polynucleotide mimetic that has been shown to have excellent hybridization properties, is referred to as a peptide nucleic acid (PNA). In PNA compounds, the sugar-backbone of a polynucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., (Science, 1991, 254, 1497-1500).

Most preferred embodiments of the invention are polynucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are polynucleotides having morpholino backbone structures of the above-referenced U.S. Pat. No. 5,034,506.

Modified polynucleotides may also contain one or more substituted sugar moieties. Preferred polynucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙ—O]ₘ—CH₃, O(CH₂)ₙ—OCH₃, O(CH₂)ₙ—NH₂, O(CH₂)ₙ—CH₃, O(CH₂)ₙ—ONH₂, and O(CH₂)ₙ—ON[(CH₂)ₙ—CH₃)]₂, where n and m are from 1 to about 10. Other preferred polynucleotides comprise one of the following at the 2′ position: C₁ to C₁₀ lower alkyl substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. A preferred modification includes 2′-methoxyethoxy (2′—O—CH₂ CH₂—OCH₃, also known as 2′—O—(2-methoxyethyl) or 2′-MOE) (Martin et al., Helv. Chim. Acta, 1995, 78, 486-504) i.e., an alkoxyalkoxy group.

Other preferred modifications include 2′-methoxy (2′—O—CH₃), 2′-aminopropoxy (2′—OCH₂ CH₂ CH₂ NH₂) and 2′-fluoro (2′-F). Similar modifications may also be made at other positions on the polynucleotide, particularly the 3′ position of the sugar on the 3′ terminal nucleotide or in 2′-5′ linked polynucleotides and the 5′ position of the 5′ terminal nucleotide. Polynucleotides may also have sugar mimetics such as cyclobutyl moieties in place of the pentofuranosyl sugar. Representative United States patents that teach the preparation of such modified sugar structures include, but are not limited to, U.S. Pat. Nos: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920, each of which is herein incorporated by reference.

Polynucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, Kroschwitz, J. I., ed. John Wiley & Sons, 1990, 858-859, those disclosed by Englisch et al., (Angewandte Chemie, IE, 1991, 30, 613), and those disclosed by Sanghvi, Y. S., (Antisense Research and Applications, 15, 289-302), and Crooke, S. T. and Lebleu, B., ed., (CRC Press, 1993). Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine 5n-methylcytosine substitutions have been shown to increase nucleic acid duplex stability by 0.6-1.2° C. (Sanghvi Y. S., Crooke, S. T. and Lebleu, B., eds., Antisense Research and Applications 1993, 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat Nos.: 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,681,941; and 5,750,692, each of which is herein incorporated by reference.

Another modification of the polynucleotides of the invention involves chemically linking to the polynucleotide one or more moieties or conjugates which enhance the activity, cellular distribution or cellular uptake of the polynucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let, 1993, 3, 2765-2770), a thiocholesterol (Oberhauser et al., Nucl. Acids Res. 1992, 20, 533-538), an aliphatic chain, e.g., dodecandiol or undecyl residues (Saison-Behmoaras et al., EMBO J., 1991, 10, 1111-1118; Kabanov et al., FEBS Lett, 1990, 59, 327-330; Svinarchuk et al., Biochimie, 1993, 75, 49-54), a phospholipid, e.g., di-hexadecyl-rac-glycerol or trimethylammonium 1,2-di-O-hexadecyl-tac-glycero-3-H-phosphonate (Manoharan et al., Tetrahedron Lett, 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

Representative United States patents, that teach the preparation of such polynucleotide conjugates include, but ate not limited to, U.S. Pat Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203; 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941, each of which is herein incorporated by reference.

It is not necessary for all positions in a given compound to be uniformly modified, and in fact more than one of the aforementioned modifications may be incorporated in a single compound or even at a single nucleoside within a polynucleotide. The present invention also includes antisense compounds which are chimeric compounds. “Chimeric” antisense compounds or “chimeras,” in the context of this invention, ate antisense compounds, particularly polynucleotides, which contain two or more chemically distinct regions, each made up of at least one monomer unit, i.e., a nucleotide in the case of a polynucleotide compound. These polynucleotides typically contain at least one region wherein the polynucleotide is modified so as to confer upon the polynucleotide increased resistance to nuclease degradation, increased cellular uptake, and/or increased binding affinity for the target nucleic acid. An additional region of the polynucleotide may serve as a substrate for enzymes capable of cleaving RNA:DNA or RNA:RNA hybrids. Consequently, comparable results can often be obtained with shorter polynucleotides when chimeric polynucleotides are used, compared to phosphorothioate deoxypolynucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more polynucleotides, modified polynucleotides, oligonucleosides and/or polynucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare polynucleotides such as the phosphorothioates and alkylated derivatives. The antisense compounds of the invention are synthesized in vitro and do not include antisense compositions of biological origin, or genetic vector constructs designed to direct the in vivo synthesis of antisense molecules.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the polynucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al. J. of Pharma Sci, 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts ate the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartic acid, lactic acid, oxalic acid, gluconic acid, glucaric acid, glucuronic acid, citric acid, benzoic acid, cinnamic acid, mandelic acid, salicylic add, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic add, ethanesulfonic add, 2-hydroxyethanesulfonic acid, ethane-1,2-disulfonic acid, benzenesulfonic add, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For polynucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric add, hydrobromic acid, sulfuric acid, phosphoric acid, nitric acid and the like; (c) salts formed with organic adds such as, for example, acetic acid, oxalic acid, tartaric add, succinic acid, maleic acid, fumaric acid, gluconic acid, citric add, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder associated with Mycobacterium tuberculosis infection which can be treated by modulating the expression of a Mycobacterium tuberculosis gene such as glutamine synthetase is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, for example.

The antisense compounds of the invention are useful for research and diagnostics, because these compounds hybridize to nucleic acids encoding, for example, glutamine synthetase, enabling sandwich and other assays to easily be constructed to exploit this fact Hybridization of the antisense polynucleotides of the invention with a nucleic acid encoding glutamine synthetase can be detected by means known in the art. Such means may include conjugation of an enzyme to the polynucleotide, radiolabelling of the polynucleotide or any other suitable detection means. Kits using such detection means for detecting the level of glutamine synthetase in a sample may also be prepared.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Polynucleotides with at least one 2′-O-methoxyethyl modification ate believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable. Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions and/or formulations comprising the polynucleotides of the present invention may also include penetration enhancers in order to enhance the alimentary delivery of the polynucleotides. Penetration enhancers may be classified as belonging to one of five broad categories, i.e., fatty acids, bile salts, chelating agents, surfactants and non-surfactants (Lee et al., Critical Reviews in Therapeutic Drug Carrier Systems, 1991, 8, 91-192; Muranishi, Critical Reviews in Therapeutic Drug Carrier Systems, 1990, 7, 1-33). One or more penetration enhancers from one or more of these broad categories may be included.

Various fatty adds and their derivatives which act as penetration enhancers include, for example, oleic acid, lauric acid, capric acid, myristic acid, palmitic acid, stearic acid, linoleic add, linolenic acid, dicaprate, tricaprate, recinleate, monoolein (a.k.a. 1-monooleoyl-rac-glycerol), dilaurin, caprylic acid, arachidonic-acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee et al., Critical Reviews in Therapeutic Drug Carrier Systems, 1991, 8, 91-192; Muranishi, Critical Reviews in Therapeutic Drug Carrier Systems, 1990, 7, 1-33; El-Hariri et al., J. Pharm. Pharmacol., 1992, 44, 651-654). Examples of some presently preferred fatty acids are sodium caprate and sodium laurate, used singly or in combination at concentrations of 0.5 to 5%.

### TYPICAL EMBODIMENTS OF THE INVENTION

As disclosed herein, we have utilized antisense polynucleotides to modulate the expression and function of M. tuberculosis genes including glutamine synthetase, aroA, ask, groES and the genes of the Antigen 85 family or complex. In this context, we demonstrate the feasibility of utilizing antisense PS-ODNs as antimicrobial agents against this pathogen. For example, we disclose that modified antisense ODNs, in which all internucleoside linkages are phosphorothioates (PS-ODNs), PS-ODNs substantially inhibit the expression of M. tuberculosis glutamine synthetase, and that the reduction in enzyme activity correlates with a reduction in the amount of the poly-L-glutamate/glutamine structure in the mycobacterial cell wall and with substantial inhibition of bacterial replication. These observations support a model of entry of the antisense PS-ODNs into the bacterial cytoplasm.

This disclosure demonstrates the feasibility of using antisense PS-ODN technology to combat M. tuberculosis, an organism whose cell wall presents a formidable battier to the entry of most biomolecules. The effect of the antisense PS-ODNs on M. tuberculosis was highly specific. Whereas the antisense PS-ODNs inhibited the growth of M. tuberculosis and the expression of its glutamine synthetase, they had no effect on M. smegmatis growth or the expression of its closely related glutamine synthetase. Moreover, control PS-ODNs had no effect on M. tuberculosis growth or glutamine synthetase expression.

Prior to this invention, it was thought that antisense polynucleotides such as phosphorothioate modified (PS-ODNs) were ineffective against mycobacteria unless a strategy was employed to afford uptake of the PS-ODNs into mycobacteria. Hence, PS-ODNs were thought to be ineffective unless either used in combination with antibiotics that “soften up the wall” of the bacteria or covalently linked to a molecule such as D-cycloserine or biotin to promote uptake (Rapaport et al., 1996. Proc. Natl. Acad. Sci. USA 93:709-713). Out finding that PS-ODNs enter M. tuberculosis without employing strategies necessary to overcome the problems observed with M. smegmatis was therefore surprising and unexpected. Moreover a comparison of the data presented herein with previously reports that oligomers complementary to M. smegmatis genes other than those targeted to the M. smegmatis ask-asd operon were ineffective provides evidence that M. smegmatis provides a limited comparative model for evaluating antisense molecules in M. tubercalosis (Rapaport et al., P.N.A.S. 93: 709-713 (1996)).

While practitioners utilizing antisense polynucleotides in eukaryotic systems have encountered some difficulties in the modulation of protein expression, skilled artisans in this field understand the fundamental differences between the cellular machinery of eukaryotes such as mammalian cells and that of prokaryotes such as M. tuberculosis. Pertinent differences between eukaryotes and prokaryotes include the significant complexity of eukaryotic mRNAs due to the introns, exons and corresponding splicing events that are not present in prokaryotic organisms. Moreover, the successful use of antisense polynucleotides to inhibit the expression of various M. tuberculosis proteins as shown in the Examples below is consistent with the observation that naturally occurring antisense polynucleotides have been found to function in prokaryotic cells as natural modulators of some proteins (see e.g. Pestka, S. Ann NY Acad Sci 660: 251-262 (1992; Simons, R. Gene 72(1-2): 35-44 (1988).

Prior to this invention, it was not known that antisense polynucleotides directed against the mRNA of the gene encoding M. tuberculosis glutamine synthetase could inhibit the growth of this pathogen. We have demonstrated that three different PS-ODNs directed against the mRNA of M. tuberculosis glutamine synthetase substantially inhibit the growth of this pathogen. We have also demonstrated that the inhibitory capacity was related to the propensity of the polynucleotide to remain in a linear configuration, i.e. the polynucleotides with the stronger propensity to remain in a linear configuration inhibited more effectively.

Prior to this invention, it was not known that antisense polynucleotides directed against the mRNA or DNA of the M. tuberculosis aroA gene could inhibit the growth of this pathogen. We have demonstrated that a PS-ODN directed against the mRNA of the M. tuberculosis aroA gene substantially inhibits the growth of this pathogen. However, not all PS-ODNs directed against the mRNA or DNA of the M. tuberculosis aroA gene inhibited M. tuberculosis growth. In our experiments, only one of four PS-ODNs directed against the mRNA or DNA of the aroA gene inhibited growth. This PS-ODN was the only one of the four that was complementary only to the coding region of the gene; the others were complementary exclusively or partly to upstream non-coding regions. The one PS-ODN that was inhibitory for M. tuberculosis growth also was one of two PS-ODNs with a strong propensity to remain linear.

Prior to this invention, it was not known that antisense polynucleotides directed against the mRNA or DNA of the M. tuberculosis ask gene could inhibit the growth of this pathogen. We have demonstrated that two PS-ODNs directed against the mRNA or DNA of the M. tuberculosis ask gene substantially inhibit the growth of this pathogen. One of the PS-ODNs was a 27-met and the other was a 36-met, both directed against the same target site (i.e. one was a 9-met extension of the other). The longer PS-ODN inhibited growth better than the shorter PS-ODN in the one experiment in which the two PS-ODNs were tested.

Prior to this invention, it was not known that antisense polynucleotides directed against the mRNA or DNA of the genes encoding the M. tuberculosis 30/32 kDa (Antigen 85) extracellular protein complex [30, 32A, 32B extracellular proteins (Antigens 85B, 85A, and 85C respectively)] could inhibit the growth of this pathogen. We have demonstrated that a PS-ODN directed against each mRNA or DNA of the genes encoding the M. tuberculosis 30/32 kDa (Antigen 85) extracellular protein complex partially inhibits the growth of this pathogen. The PS-ODN was fully complementary to the mRNA encoding the 32A protein and mismatched by one nucleotide each with the mRNAs encoding the 30 or 32B proteins.

The data presented herein which shows that antisense polynucleotides can be used to inhibit the expression of a variety of different M. tuberculosis genes provides evidence that neither the structure of the prokaryotic M. tuberculosis cell wall nor the M. tuberculosis cellular machinery provide significant barriers to the use of antisense polynucleotides to modulate the expression of M. tuberculosis genes. Instead, the data presented herein provides evidence that the physiology of this organism is particularly amenable to the use antisense polynucleotides for modulating protein expression in the manner described herein.

As noted above, a variety of permutations of the antisense technology utilized herein are well known in the art, as disclosed for example in WO9803533A1, WO9950277A1, WO9604788A1, WO9500638A3, W09104753A1 and WO9901579A1 all which are incorporated herein by reference. The invention disclosed herein provides not just new antibiotics to treat both drug resistant and drug sensitive strains but a whole new approach to treatment of M. tuberculosis infection. The technology allows the generation of polynucleotides directed against thousands of different mRNA or DNA targets. Moreover, the polynucleotides could be used in combination, as we have found that combinations of different polynucleotides are more potent than individual ones. Moreover, provided they are sufficiently long to hybridize with the target nucleic acid, the polynucleotides could have one or several mismatches with the target nucleic add and still be efficacious; hence, they would tolerate some genetic diversity among strains and some mutations of the target nucleic add. Moreover, modifications of internucleoside phosphates other than phosphorothioates may be used to inhibit the replication of M. tuberculosis. Without comprising a complete listing, but as examples, one may mention methylphosphonates, phosphoroboronates, phosphoromorpholidates, butyl amidated, and peptide nucleic acid linkages. There may also be changes in the sugar ring, such as 2′-0 methyl or methoxy ethoxy additions to the 2′-ribosyl ring. There may also be modifications of the purine and pyrimidine bases themselves. 5 The antisense molecules discussed in detail in the Examples 1-7 below are directed to the glutamine synthetase (L-glutamate:ammonia ligase (ADP-forming); EC 6.3.1.2) gene, which we recently identified as an important determinant of M. tuberculosis pathogenesis (see e.g. Harth et al., (1994) Proc. Natl. Acad. Sci. USA 91, 9342-9346; Harth et al., (1997) J. Biol. Chem. 272, 22728-22735 and Harth et al., (1999) J. Exp. Med. 189, 1425-1435). An enzyme that plays a central role in nitrogen metabolism in every cell, glutamine synthetase is one of 10 proteins released in large quantity into the bacterium's extracellular milieu, whether the bacterium is growing axenically or intraphagosomally in human mononuclear phagocytes, the primary host cells (see e.g. Harth et al., (1994) Proc. Natl. Acad. Sci. USA 91, 9342-9346). Of great interest, extracellular release of glutamine synthetase is unique to pathogenic mycobacteria and correlated with the presence of a poly-L-glutamate/glutamine component in the cell wall of these organisms, suggesting that the enzyme is not only involved in the synthesis of this heteropolymer but also that its presence is significant to virulence (Harth et al., (1999) J. Exp. Med. 189, 1425-1435). Consistent with this concept, treatment of bacteria with L-methionine-S-sulfoximine, an irreversible inhibitor of glutamine synthetase, results in a decrease in extracellular glutamine synthetase activity, a marked reduction in the amount of the poly-L-glutamate/glutamine heteropolymer in the mycobacteria cell wall, and inhibition of bacterial growth both in broth culture and in human macrophages (Harth et al., (1999) J. Exp. Med. 189, 1425-1435).

Antisense oligodeoxyribonucleotides (ODNs), which can base pair with a gene's transcript, constitute a new technology for the control of gene expression in prokaryotes and eukaryotes, including mammalian cells (Zamecnik et al., (1978) Proc. Natl. Acad. Sci. USA 75, 280-284 and Stephenson et al., (1978) Proc. Natl. Acad. Sci. USA 75, 285-288). In addition, this technology shows promise as a means for developing new chemotherapeutic agents against human diseases (Zamecnik et al., Antisense Nucleic Acid Drug Dev. (1997) 7, 199-202). With regard to the development of new antibiotics, antisense ODNs have been used in vitro to inhibit the replication of such pathogens as Plasmodium falciparum, Toxoplasma gondii, and HIV (Barker et al., (1996) Proc. Natl. Acad. Sci. USA 93, 514-518; Nakaar et al., (1999) J. Biol. Chem. 274, 5083-5087 and Lisziewicz et al., (1992) Proc. Natl. Acad. Sci. USA 89, 11209-11213), and an antisense PS-ODN developed for the treatment of cytomegalovirus retinitis in AIDS patients, became the first antisense ODN-based drug approved for human use by the U.S. Food and Drug Administration (Anderson et al., (1996) Antimicrobial. Agents Chemother. 40, 2004-2011).

In the invention disclosed herein, an mRNA or DNA target of the M. tuberculosis genome is selected. Typically, one can focus on nucleic adds coding for proteins that are constitutively expressed, produced in high quantity, or involved in an essential aspect of bacterial metabolism or cell wall synthesis. Polynucleotides are synthesized that are complementary to these targets and at the same time, not homologous to their human counterpart if any. These polynucleotides are then administered to a person infected with M. tuberculosis.

While a variety of polynucleotides can effect the results provided herein, preference is given to the following polynucleotides that have the strongest propensity to remain in a linear configuration since, in the case of the mRNA of glutamine synthetase, the inhibitory capacity was directly related to the propensity of the polynucleotide to remain in a linear configuration. Such a propensity to remain in a linear configuration can be determined by a number of methods known in the art such as by using the SIGMA-GENOSYS OLIGO-5 SECONDARY STRUCTURE ANALYSIS PROGRAM. Moreover, preference is given to polynucleotides that are complementary to the coding region of the protein since, in the case of the M. tuberculosis aroA gene, only the one PS-ODN of four that was complementary to the coding region was inhibitory of M. tuberculosis growth. In addition, preference is given to polynucleotides that are relatively long since in the case of the ask gene, where we studied two PS-ODNs with the same target region, the longer PS-ODN (a 9-mer extension of the other PS-ODN), was more inhibitory.

The glutamine synthetase antisense PS-ODNs disclosed herein exhibit three simultaneous effects on M. tuberculosis: reduction in intracellular and extracellular glutamine synthetase activity, reduction in the formation of the poly-L-glutamate/glutamine cell wall structure, and inhibition of bacterial growth. Except that it acts almost exclusively on extracellular glutamine synthetase, the glutamine synthetase inhibitor L-methionine-S-sulfoximine exerts parallel effects on M. tuberculosis. On the basis of studies with this inhibitor (Harth et al., (1999) J. Exp. Med. 189, 1425-1435), we have postulated that the reduction in the formation of the poly-L-glutamate/glutamine heteropolymer is a consequence of reduced extracellular glutamine synthetase activity. While not being bound to any specific theory, it seems possible that the PS-ODNs reduce the amount of the heteropolymer by the same mechanism. The reduction in the heteropolymer in turn likely affects mycobacterial growth by disturbing the integrity of the cell wall. In addition, the recent observations that poly-L-glutamine degradation in mammalian cells results in intracellular protein aggregation raises the possibility of a novel mechanism by which M. tuberculosis might subvert host cell function—release of portions of its cell wall poly-L-glutamate/glutamine heteropolymer (Green, H. (1993) Cell 74, 955-956; Karpuj et al., (1999) Proc. Natl. Acad. Sci. USA 96, 7388-7393 and Kazantsev et al., (1999) Proc. Natl. Acad. Sci. USA 96, 11404-11409).

Although the effects of the glutamine synthetase antisense PS-ODNs on M. tuberculosis were highly consistent, they were not as efficacious as L-methionine-S-sulfoximine (Harth et al., (1999) J. Exp. Med. 189, 1425-1435). At 2 μM, the latter compound inhibited bacterial growth by 1.5-2 log units, and at 20 μM, it reduced bacterial growth by ≧2.5 log units, extracellular glutamine synthetase activity by 80%, and the amount of the poly-L-glutamate/glutamine cell wall structure by 93%. In contrast, the combination of all 3 PS-ODNs at a concentration of 10 μM each, the most inhibitory preparation, reduced bacterial growth by only 1.25 logs, extracellular glutamine synthetase activity by only 48%, and the amount of the heteropolymer by only 24%. Even if inhibition is incomplete, the use of concentrations of PS-ODNs higher than 10 μM is generally not as desirable since, as the concentration of PS-ODNs is increased, non-sequence specific interactions with proteins and nucleic acids rise, particularly the polyanionic effects marker et al., (1996) Proc. Natl. Acad. Sci. USA 93, 514-518), (Stein C. A. & Chen, Y -C. (1993) Science 261, 1004-1012 and Agrawal et al., (1998) Curr. Opin. Chemical Biol., 2, 519-528).

Two potential explanations for the observed effect of the glutamine synthetase antisense PS-ODNs in our study are inefficient uptake across the mycobacterial cell wall and intracellular instability. With respect to uptake, both strategies to improve PS-ODN uptake were unsuccessful. We first attempted to target the PS-ODN to ribosomes by tethering the PS-ODN to an amikacin moiety. Such PS-ODNs were not mote inhibitory than free PS-ODNs. Since tie amikacin moiety lost its antibiotic activity as a result of being linked to the PS-ODNs, it also may have lost its ability to interact in a positive way with components of the mycobacterial cell wall and mycobacterial ribosomes. We also attempted to improve uptake of PS-ODNs by “softening” the mycobacterial cell wall with antibiotics. In future studies, we hope to improve transport of PS-ODNs across the mycobacterial cell wall by using alternative transporters such as a covalently linked ethambutol residue (Rapaport et al., (1996) Proc. Natl. Acad. Sci. USA 93, 709-713).

Although the experiments were not designed to investigate rigorously the effect of mismatches in the PS-ODNs, the data suggested that the antisense PS-ODNs had to be highly specific for their target site. PS-ODNs perfectly matched with the M. tuberculosis glutamine synthetase mRNA transcript but mismatched at 2-4 nucleotide positions with the M. smegmatis glutamine synthetase transcript inhibited expression of the recombinant M. tuberculosis enzyme but not the endogenous M. smegmatis enzyme in M. smegmatis. However, one mismatch was tolerated since a PS-ODN mismatched at one nucleotide with the M. tuberculosis mRNA transcript inhibited both expression of the enzyme in M. tuberculosis and bacterial growth to the same extent as its perfectly matched PS-ODN counterpart.

Our disclosure demonstrates that antisense PS-ODNs can add to the inhibitory effect of conventional antibiotics. In the presence of subinhibitory concentrations of ethambutol and/or polymyxin B nonapeptide, antisense PS-ODNs provided an incremental increase in inhibition of M. tuberculosis growth. Tuberculosis is typically treated with a cocktail of antibiotics to prevent the emergence of resistant organisms, which arise at a frequency of 10⁶ to 10⁷ to conventional drugs. Although in this study, because of safety considerations, we demonstrated an effect of antisense PS-ODNs against a drug-sensitive M. tuberculosis strain, it is likely that the PS-ODNs also would be effective against drug-resistant strains since resistance mechanisms are generally antibiotic specific and PS-ODNs differ substantially from conventional antibiotics.

Pharmacokinetic studies by Agrawal and colleagues indicate that therapeutic concentrations of ODNs are achievable in blood and tissues at nontoxic doses (Agrawal et al., (1998) Curr. Opin. Chemical Biol. 2, 519-528 and Agrawal et al., (1997) Ciba Foundation Symposium 209, 60-78). Although the plasma concentrations of injected or orally administered PS-ODNs or ODNs with other modifications diminish rapidly with time, measured in minutes or hours, tissue concentrations remain relatively high for several days. With respect to tuberculosis, it is noteworthy that when fluorescein isothiocyanate labelled ODNs are administered intravenously to mice, the ODNs ate preferentially taken up by mononuclear phagocytes (Zhao et al, (1998) Antisense Nucleic Acid Drug Dev. 8, 451-458), the host cells for M. tuberculosis. Thus, our disclosure and the pharmacologic work of Agrawal and colleagues (Agrawal et al., (1998) Curr. Opin. Chemical Biol. 2, 519-528, Agrawal et al., (1997) Ciba Foundation Symposium 209, 60-78 and Zhao et al., (1998) Antisense Nucleic Acid Drug Dev. 8, 451-458) suggests the feasibility of employing modified nuclease resistant ODNs as part of a cocktail of antituberculous drugs.

Antisense polynucleotides against mRNA or DNA targets of M. tuberculosis can be administered to people with active tuberculosis or people harboring M. tuberculosis in a latent state as evidenced by a positive diagnostic test for this organism. Appropriately formulated, the polynucleotides can be administered by any number of routes such as intravenously, intramuscularly, intraperitoneally, subcutaneously, orally, etc. The polynucleotides can inhibit the growth of the M. tuberculosis and thereby treat active tuberculosis or prevent latent tuberculosis from reactivating. Since the polynucleotides were particularly effective against M. tuberculosis entering the stationary phase of growth in vitro, treatment with PS-ODNs may be particularly efficacious against latent infection with M. tuberculosis.

The invention described herein encompasses a variety of embodiments. A typical illustrative embodiment consists of a method for inhibiting Mycobacterium tuberculosis glutamine synthetase protein expression by contacting a Mycobacterium tuberculosis bacterium with an effective amount of an antisense polynucleotide that hybridizes to a Mycobacterium tuberculosis glutamine synthetase polynucleotide, wherein the antisense polynucleotide hybridizes to a region of the Mycobacterium tuberculosis glutamine synthetase polynucleotide encoding the glutamine synthetase protein, thereby inhibiting Mycobacterium tuberculosis glutamine synthetase protein expression. An effective amount of the therapeutic composition is determined based on the intended goal, in this context, the inhibition of protein expression. Alternatively the goal may be to contact Mycobacterium tuberculosis with an effective amount of an antisense polynucleotide capable of inhibiting the proliferation of Mycobacterium tuberculosis. Means for determining an effective amount sufficient to achieve a goal such as the inhibition of protein expression or the inhibition of bacterial growth are well known in the art and typical assays to measure such factors are provided in the examples below. The quantity to be administered, both according to number of treatments and unit dose, depends on the protection desired. Precise amounts of the therapeutic composition also depend on the judgment of the practitioner and are peculiar to each individual. Factors affecting dose include physical and clinical state of the patient, the route of administration and the potency, stability and toxicity of the particular therapeutic substance.

In preferred embodiments of the invention, the antisense polynucleotide has a modification to its internucleoside phosphates linkages such as phosphorothioates, methylphosphonates, phosphoroboronates, phosphoromorpholidates, butyl amidates, and peptide nucleic add linkages. A number of representative polynucleotides targeting different regions of selected Mycobacterium tuberculosis genes include 5′-GAC GTC GTC GGG CGT CTT-3′ (SEQ ID NO: 18), 5′-CAT GCC GGA CCC GTT GTC GCC-3′ (SEQ ID NO: 19) and 5′-CCA CAG CGA CTG ATG ACA GTG CAT-3′ (SEQ ID NO: 20) which are specific for glutamine synthetase.

Another embodiment of the invention consists of contacting the Mycobacterium tuberculosis with an effective amount of a second antisense polynucleotide that hybridizes to a Mycobacterium tuberculosis glutamine synthetase polynucleotide, wherein the second antisense polynucleotide hybridizes to a region of the Mycobacterium tuberculosis glutamine synthetase polynucleotide encoding the glutamine synthetase protein that is distinct from the region targeted by the first antisense oligonucleotide. Alternatively one can contact the Mycobacterium tuberculosis with an effective amount of a second antisense polynucleotide that hybridizes to a different Mycobacterium tuberculosis polynucleotide, for example an mRNA or gene selected from the group consisting of aroA, ask, groES and the genes of the Antigen 85 complex.

Yet another embodiment consists of combination therapy as is known in the art, for example contacting the Mycobacterium tuberculosis with an effective amount of an antibiotic capable of inhibiting the proliferation of Mycobacterium tuberculosis. In typical illustrative embodiments, the antibiotic is selected from the group consisting of rifampin, isoniazid, amikacin, ethambutol and polymyxin B nonapeptide.

Yet another embodiment consists of an antisense compound of about 15 to about 50 nucleobases in length targeted to a portion of a nucleic acid molecule encoding a Mycobacterium tuberculosis protein such as the glutamine synthetase protein, wherein the antisense compound specifically hybridizes with the nucleic acid molecule encoding the protein, thereby inhibiting expression of the protein. Typically the antisense compound is an antisense oligonucleotide such as one of the antisense polynucleotides provided herein. As noted above, preferably the antisense polynucleotide has at least one modified internucleoside linkage such as a phosphorothioate linkage. In addition, it is preferable that the antisense polynucleotide has a complete homology with the nucleic acid molecule encoding the Mycobacterium tuberculosis glutamine synthetase protein.

Yet another embodiment of the invention consists of a process for producing an antisense compound that inhibits the expression of any Mycobacterium tuberculosis protein such as glutamine synthetase. As part of this process one typically begins by synthesizing a antisense polynucleotide (typically about 15 to about 50 nucleobases in length) which is designed to hybridize to a portion of polynucleotide encoding a protein in the Mycobacterium tuberculosis genome. As the complete genome of Mycobacterium tuberculosis has been sequenced and published (see Cole et al., (1998) Nature 393, 537-544), the skilled artisan can readily design antisense polynucleotides targeting portions of a Mycobacterium tuberculosis polynucleotide encoding a Mycobacterium tuberculosis protein in the genome. A next step in this process entails contacting a culture of Mycobacterium tuberculosis with an effective amount of the antisense polynucleotide under conditions such that the antisense polynucleotide hybridizes with the targeted Mycobacterium tuberculosis polynucleotide. A next step in this process entails comparing the levels of Mycobacterium tuberculosis protein expression (i.e. the protein encoded by the targeted polynucleotide) in the culture contacted with the effective amount of the antisense polynucleotide to the levels of Mycobacterium tuberculosis protein expression in a control culture of Mycobacterium tuberculosis not contacted with the antisense polynucleotide. The skilled artisan understands that protein expression in a control culture of Mycobacterium tuberculosis not contacted with an antisense polynucleotide (i.e. the normal level of the targeted protein's expression) may be predetermined so that a typical average value representing protein expression (e.g. number of molecules, activity etc.) in a control culture is used in the evaluation of potentially inhibitory molecules. A next step in this process entails determining whether the antisense polynucleotide inhibits the expression of the targeted protein by observing the level of that protein's expression in the Mycobacterium tuberculosis culture contacted with the antisense polynucleotide relative to the level of that protein's expression in the Mycobacterium tuberculosis control culture. A closely related embodiment of this invention consists of an antisense polynucleotide produced according to this process.

The embodiments of the invention as described above can be used in a variety of contexts, for example either with or without agents such as cell softening agents.

The present invention is further detailed in the following Examples, which are offered by way of illustration and are not intended to limit the invention in any manner. All patent and literature references cited in the present specification are hereby incorporated by reference in their entirety.

### EXAMPLES

### Example 1

Bacterial Cultures.

M. tuberculosis strain Erdman (ATCC 35801) and M. smegmatis 1-2c (Garbe et al., (1994) Microbiol 140, 133-138) were cultured in 7H9 medium (Difco) supplemented with 2% glucose at 37° C. M. tuberculosis was maintained in a 5% CO₂-95% air atmosphere as unshaken cultures (because of safety considerations) and M. smegmatis was maintained at ambient conditions with vigorous shaking. For studies of the effect of PS-ODNs on the bacteria, M. tuberculosis and M. smegmatis were cultured in duplicate in 1 ml, 2 ml, or 5 ml of 7H9 broth in polystyrene tubes (Fisher) or tissue culture flasks (Costar) in the presence of medium alone, PS-ODNs at final concentrations of 0.1, 1, or 10 μM, or PS-ODNs plus inhibitory and/or subinhibitory concentrations of the antibiotics amikacin (0.058, 0.58, and 5.8 μg/ml), ethambutol (0.1, 0.25, and 0.5 μg/ml), or polymyxin B nonapeptide (0.1, 0.25, 0.5 μg/ml). All antibiotics were from Sigma. The minimal inhibitory concentrations (MIC) of the antibiotics for M. tuberculosis Erdman and M. smegmatis 1-2c were established in bacterial cultures grown under the same conditions in the presence of antibiotics ranging in concentration from 0.01-32 μg/ml for amikacin and 0.1-25 μg/ml for ethambutol and polymyxin B nonapeptide.

### Example 2

Glutamine Synthetase PS-ODN Selection and Preparation.

Three target sites for the binding of the antisense PS-ODNs were chosen (Table 1). One site, located near the 5′ end of the glutamine synthetase mRNA, corresponds to codons 4-9 of the glutamine synthetase mRNA open reading frame, starting from the first codon GTG which specifies the initiator methionine residue. The other two sites, located in the vicinity of a catalytically important histidine residue (H276), correspond to codons 269-275 and 275-282, respectively. Antisense PS-ODNs were synthesized on a 394 DNA/RNA synthesizer (Applied Biosystems) using standard phosphoroamidite chemistry. Phosphorothioate bonds were introduced by oxidation with the Beaucage thiolating reagent (Padmapriya et al., (1994) Antisense Res. Develop. 4, 185-199) and assembled PS-ODNs were purified by HPLC and lyophilized. Amikacin derivatives were synthesized by a phosphorothioate based methodology, linking the antibiotic via one of its amino groups to a phosphate residue at the end of a 3′ attached 18-atom spacer arm. PS-ODN stock solutions were prepared just prior to their use and added to mycobacterial cultures after filter sterilization through 0.45 μm HT Tuffryn membrane filters (Gelman Sciences).

Selection of PS-ODN targets and stoichiometric considerations. M. tuberculosis contains four genetic loci with domains that exhibit homologies to glutamine synthetase genes from other bacteria (see e.g. Harth et al., (1994) Proc. Natl. Acad. Sci. USA 91, 9342-9346 and Cole et al., (1998) Nature 393, 537-544). However, in the Erdman strain, and presumably other clinical strains, only one gene, designated glnA1 and located at map position Rv2220 of the M. tuberculosis H37Rv genome (Cole et al., (1998) Nature 393, 537-544), expresses an active glutamine synthetase. This enzyme has an apparent molecular mass of ˜680,000 Da (12 subunits of ˜56,000 Da each) (see e.g. Harth et al., (1994) Proc. Natl. Acad. Sci. USA 91, 9342-9346). It is not known if the other loci (glnA2, glnA3, glnA4) are expressed. M. tuberculosis and other pathogenic mycobacteria of the tuberculosis complex express large amounts of the enzyme and export a large proportion of what is produced—one-third in the case of M. tuberculosis (see e.g. Harth et al., (1994) Proc. Natl. Acad. Sci. USA 91, 9342-9346 and Harth et al., (1997) J. Biol. Chem. 272, 22728-22735). Nonpathogenic mycobacteria such as M. smegmatis and M. phlei typically exhibit a lower glutamine synthetase expression level than pathogenic mycobacteria and export less than 1/100th of the total glutamine synthetase produced (see e.g. Harth et al., (1994) Proc. Natl. Acad. Sci. USA 91, 9342-9346). To date, it is unknown if the nonpathogenic mycobacteria share a similar genomic arrangement of glutamine synthetase specific DNA loci.

Because the rate of transcription under standard growth conditions is nearly constant (see e.g. Harth et al., (1997) J. Biol. Chem. 272, 22728-22735) and because the downstream effects of inhibition by antisense PS-ODNs, i e. a decrease in the amount of detectable enzyme and its activity, can be tracked continuously and quantitatively, the glutamine synthetase transcript is a logical target to study regulation of gene expression by antisense PS-ODNs.

The glutamine synthetase coding region contains 1,434 base pairs, excluding the stop codon. The primary transcript under standard axenic growth conditions is 1,500-1,600 nucleotides in length (see e.g. Harth et al., (1997) J. Biol. Chem. 272, 22728-22735). Based on densitometric analyses of our Northern hybridizations, we calculated that ˜11 glutamine synthetase gene transcripts of ˜1,550 nucleotides are present pet cell.

We selected three target sites, one neat the 5′ end of the glutamine synthetase transcript and two at a region encoding catalytically important residues. All three mRNA regions ate devoid of any stable secondary structures, and they ate thus theoretically readily accessible to the complementary PS-ODNs. However, the possibility that the in vivo structure of the mRNA is much more complex due to the binding of proteins cannot be excluded. All PS-ODNs were at least 18 bases in length, making random hybridization to the M. tuberculosis genome [4.4×10⁶ base pairs (Cole et al., (1998) Nature 393, 537-544)] very unlikely; moreover, partial hybridization of short sequences within such PS-ODNs, while theoretically possible, would likely be unstable.

### Example 3

Specificity of Antisense PS-ODNs for M. tuberculosis glutamine Synthetase mRNA.

To confirm the specificity of the antisense PS-ODNs, we studied their effect on the transcript of glnA1 of M. smegmatis, a closely related gene. A comparison of the M. tuberculosis and M. smegmatis glutamine synthetase genes revealed a very high degree of DNA sequence identity and an overall DNA sequence similarity of ˜70% and ptotein similarity of ˜80%. At the target sites, nucleotide differences between the two mycobacterial species ate sufficient so as to anticipate that the antisense PS-ODNs, based on the DNA sequence of the M. tuberculosis Erdman glutamine synthetase gene, would bind only to the M. tuberculosis gene (Table 1). The aligned RNA sequences demonstrate that the target site for PS-ODN #4-9 is different in 3 nucleotide positions (5′-AAG ACG CCC GAC GAC GUC-3′ (SEQ ID NO: 1) for M. tuberculosis and 5′-AAG ACG UCG GAC GAC AUC-3′ (SEQ ID NO: 4) for M. smegmatis), the target site for PS-ODN #269-275 is different in 2 nucleotide positions (5′-GGC GAC AAC GGG UCC GGC AUG-3′ (SEQ ID NO: 2) for M. tuberculosis and 5′-GGC GAC AAC GGU UCG GGC AUG-3′ (SEQ ID NO: 5) for M. smegmatis), and the target site for PS-ODN #275-282 is different in 4 nucleotide positions (5′-AUG CAC UGU CAU CAG UCG CUG UGG-3′ (SEQ ID NO: 3) for M. tuberculosis and 5′-AUG CAC GCC CAC CAG UCG CUG UGG-3′ (SEQ ID NO: 6) for M. smegmatis). To compare the effect of the antisense PS-ODNs on M. smegmatis and M. tuberculosis glutamine synthetase mRNA, we added the PS-ODNs to cultures of the M. smegmatis 1-2c wildtype strain and its recombinant isotype expressing the M. tuberculosis Erdman glutamine synthetase from a mycobacterial shuttle vector (Harth et al., (1997) J. Biol Chem. 272, 22728-22735). We set up the cultures as described above for M. tuberculosis cultures, except that we added the PS-ODNs to M. smegmatis cultures after two days and monitored the cultures for the subsequent 4 days, by which time they had reached stationary phase. We were able to readily differentiate the recombinant and endogenous glutamine synthetases in the recombinant strain on the basis of N-terminal amino acid analysis, as the first amino acid residue of each of the 12 identical enzyme subunits is a threonine in M. tuberculosis and an alanine in M. smegmatis (Harth et al., (1997) J. Biol. Chem. 272, 22728-22735). Moreover, virtually all extracellular enzyme activity in recombinant M. smegvatis cultures is exported recombinant M. tuberculosis glutamine synthetase (Harth et al., (1997) J. Biol. Chem., 272, 22728-22735).

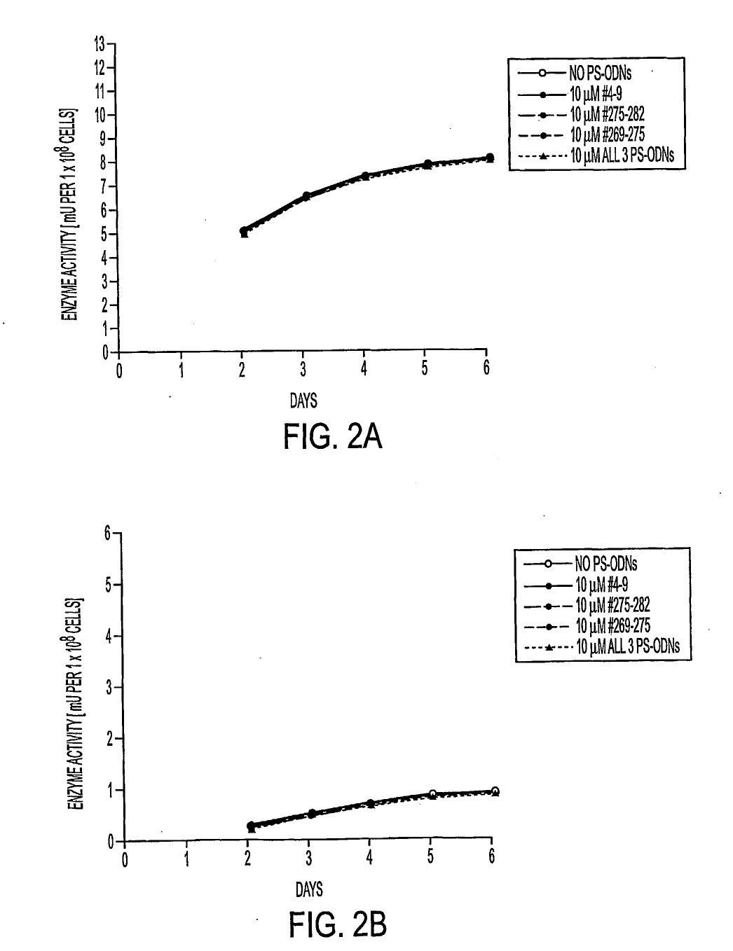

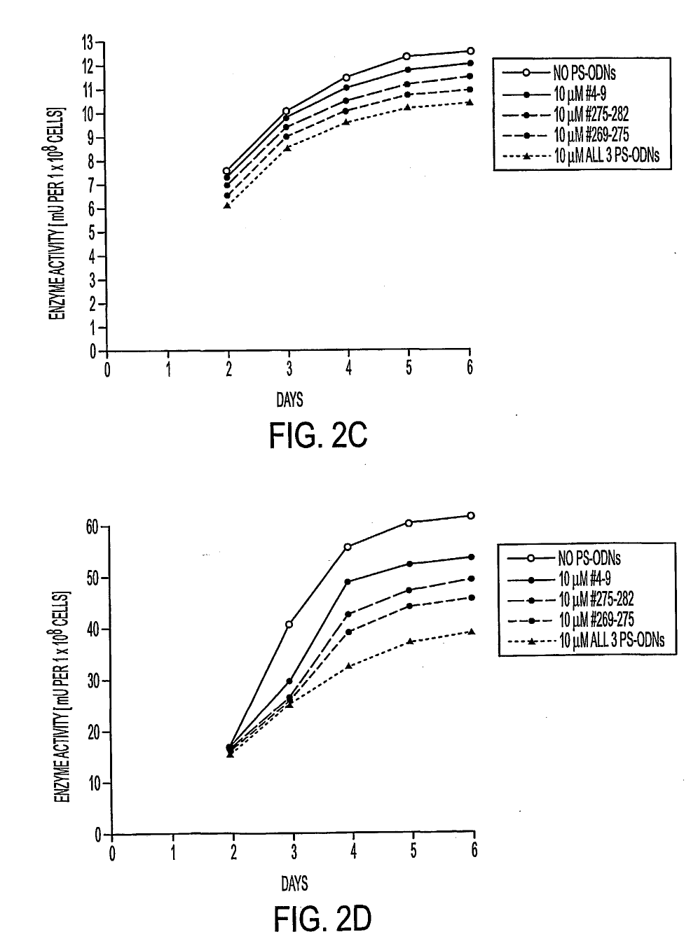

The results of these experiments can be summarized as follows (FIG. 2): (i) At 10 μM antisense PS-ODNs perfectly matched with the recombinant M. tuberculosis transcript but mismatched at 2-4 nucleotide positions with the endogenous M. smegmatis transcript), the cellular glutamine synthetase activity in the parent strain remained essentially unchanged at 7.2 mU per 1×10⁸ cells when compared with uninhibited cultures (FIG. 2A). Neither the various combinations of 2 PS-ODNs nor the addition of all 3 PS-ODNs decreased the detectable cellular enzyme activity. Similarly, extracellular glutamine synthetase activity remained unchanged in the parent strain at barely detectable levels amounting to ≦0.1 mU of enzyme activity (FIG. 2B). (ii) In contrast, the cellular glutamine synthetase activity of the recombinant strain, 60% of which is endogenous M. smegmatis enzyme activity and 40% recombinant M. tuberculosis enzyme activity, decreased from 12.4 mU in uninhibited cultures to 11.7 mU for cultures growing in the presence of PS-ODN #4-9, to 11.4 mU in the presence of PS-ODN #275-282, to 11.0 mU in the presence of PS-ODN #269-275, and to 10.7 mU in the presence of all 3 PS-ODNs (FIG. 2C). Assuming the entire 1.7 mU decrease in cellular enzyme activity in the presence of 3 PS-ODNs was in the fraction of enzyme activity due to recombinant M. tuberculosis glutamine synthetase (˜5 mU in a total of 12.4 mU), this fraction of activity was decreased 34%, similat to the decrease observed in M. tuberculosis cultures. The extracellular enzyme activity of the recombinant M. smegmatis strain, of which ≧99% is due to the export of recombinant M. tuberculosis glutamine synthetase, decreased from 61.9 mU in uninhibited cultures to 55.2 mU in the presence of PS-ODN #49, to 47.3 mU in the presence of PS-ODN #275-282, to 44.6 mU in the presence of PS-ODN #269-275, and to 36.7 mU in the presence of all 3 PS-ODNs (S.D. ≦15% for all data). The 41% reduction in the presence of all 3 PS-ODNs was sriilar to the decrease observed in M. tuberculosis cultures (FIG. 2D). Again, the inhibitory capacity of each PS-ODN correlated directly with its propensity to remain in a linear conformation as described above for the M. tuberculosis cultures (Table 1). Combinations of 2 or 3 PS-ODNs at 10 μM each were somewhat better than single PS-ODNs. (iii) At all concentrations and combinations tested, the control PS-ODNs did not affect the detectable enzyme activity. (iv) The observed reduction in detectable enzyme activity in recombinant M. smegmatis (FIG. 2D) was mirrored by a reduction in the detectable amount of glutamine synthetase protein molecules. It is not known if regulation of glutamine synthetase expression in M. smegmatis is patterned according to the mechanisms governing glutamine synthetase expression in M. tuberculosis (see e.g. Harth et al., (1997) J. Biol. Chem. 272, 22728-22735).

Our finding that expression of recombinant cellular and exported M. tuberculosis glutamine synthetase is decreased in M. smegmatis to approximately the same degree as in M. tuberculosis suggests strongly that the PS-ODNs are taken up into M. smegmatis and bind to their cognate target. Our finding that the antisense PS-ODNs inhibit expression of the recombinant enzyme but not the endogenous enzyme in M. smegmatis underscores the specificity of the binding of the PS-ODNs to the M. tuberculosis enzyme transcript since they were perfectly matched with the transcript of the recombinant glutamine synthetase gene but mismatched at 2-4 nucleotide positions with the transcript of the endogenous glutamine synthetase gene.

### Example 4

Effect of PS-ODNs on glutamine Synthetase Activity and Expression.

Aliquots were removed at weekly (M. tuberculosis) or daily (M. smegmatis) intervals from the cultures and glutamine synthetase activity was assayed by the transfer assay, calibrated with the purified M. tuberculosis enzyme representing 1×10⁸ cell equivalents. An enzyme unit was defined as the amount of glutamine synthetase that produced 1 μmol of γ-glutamylhydroxamate pet min at 37° C. (see Harth et al., (1994) Proc. Natl. Acad. Sci. USA 91, 9342-9346). Proteins were analyzed by denaturing polyacrylamide gel electrophoresis followed by staining with Coomassie brilliant blue R, and protein concentrations were determined by the bicinchoninic acid reagent (Pierce). The possibility of leakage of cytoplasmic glutamine synthetase from dead or dying mycobacteria was assessed by monitoring the activity of the cytoplasmic marker protein lactate dehydrogenase during the 6 week/6 day growth period both in the culture medium and in the cell pellet, using a commercially available diagnostic kit (Sigma). Extracellular lactate dehydrogenase activity never exceeded 0.2% of intracellular activity.

Effect of the Antisense PS-ODNs on the Detectable Activity and Amount of glutamine Synthetase in M. tuberculosis Erdman.

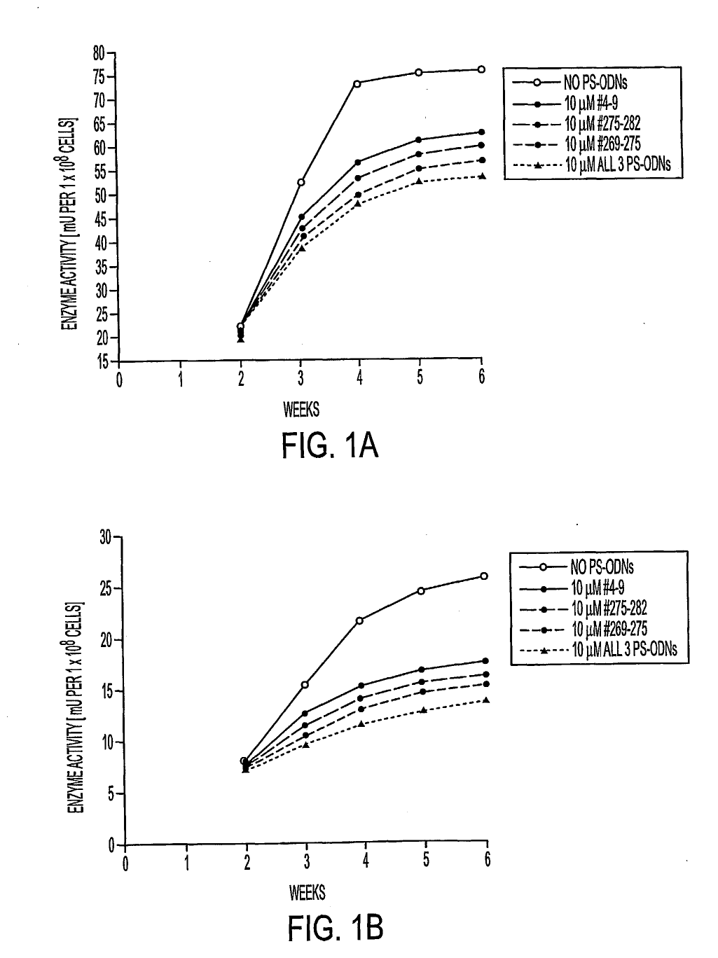

We first cultured M. tuberculosis in 1 or 2 ml of broth medium for 2 weeks to provide a sufficient number of bacteria for reliable measurements of both intra- and extracellular glutamine synthetase activity, i. e. ≧5×10⁶ cell equivalents per assay. We then inoculated the cultures with three different concentrations (0.1, 1, and 10 μM) of the following PS-ODNs: a) each antisense PS-ODN alone; b) combinations of 2 PS-ODNs; and c) the combination of all three PS-ODNs. We inoculated an identical set of cultures with antisense PS-ODNs containing amikacin tethered to the 3′ end, and a third set of cultures with all the corresponding nonsense controls (all codons mismatched at the second nucleotide position) to each of the three antisense PS-ODNs. We assayed the cultures for enzyme activity from week 2 to 6 (FIG. 1).

The results of these experiments can be summarized as follows: (i) At 10 μM antisense PS-ODN, the only effective concentration, the cellular glutamine synthetase activity, standardized for all measurements to the detectable activity pet 1×10⁸ cells, decreased at 6 weeks from 76.5 mU in uninhibited cultures to 61.3 mU for PS-ODN #4-9, 57.1 mU for PS-ODN #275-282, and 54.8 mU for PS-ODN #269-275 (S.D. ≦10% for all data). Combinations of 2 or 3 PS-ODNs at 10 μM each were slightly more inhibitory than single PS-ODNs; the combination of all 3 PS-ODNs was the most inhibitory, decreasing the activity by 33% to 51.4 mU. Interestingly, the inhibitory capacity of a PS-ODN was directly related to its propensity to remain in a linear conformation (Table 1), as assessed using the Sigma-Genosys, Inc. Oligo-5 Secondary Structure Analysis program. Whereas the most effective PS-ODN (PS-ODN #269-275) has a strong potential to maintain a linear structure, the second most effective PS-ODN (PS-ODN #275-282) has a moderate potential to do so, and the least effective PS-ODN (PS-ODN #49) has a weak potential to maintain a linear structure. (ii) In the presence of antisense PS-ODNs, extracellular glutamine synthetase activity was also decreased and to an extent somewhat greater than for the cellular enzyme activity. The activity decreased from 25.8 mU under standard growth conditions to 17.1 mU in the presence of PS-ODN #4-9, 15.6 mU in the presence of PS-ODN #275-282, and 15.1 mU in the presence of PS-ODN #269-275. Combinations of 2 PS-ODNs were not significantly more inhibitory than 1 PS-ODN; however, the combination of all 3 PS-ODNs decreased enzyme activity to 13,3 mU, a reduction of 50% below the level of an uninhibited culture (S.D. ≦10% for all data). Possibly, at these high concentrations of antisense PS-ODNs, the amount of intracellularly expressed glutamine synthetase is lowered to the point that the cell is no longer able to export the same proportion of enzyme molecules as it normally does. (iii) PS-ODNs tethered to an amikacin moiety, in theory to guide PS-ODNs to the ribosomes, had no greater inhibitory effect than PS-ODNs not tethered to amikacin in this as well as in all subsequent studies. Once tethered to a PS-ODN, the antibiotic itself was completely inactive, even though, at a PS-ODN concentration of 10 μM, it was present at a concentration (5.8 μg per ml) at which free amikacin is strongly bactericidal. Consistent with this, nonsense PS-ODNs tethered to amikacin had no growth inhibitory effect whatsoever, either at 10 μM, a concentration at which free amikacin is bactericidal, or 1 μM, a concentration at which free amikacin is bacteriostatic. (iv) At all concentrations and combinations tested, control PS-ODNs did not affect the detectable enzyme activity. (v) The observed reductions in detectable enzyme activity were mirrored by a reduction in the amount of glutamine synthetase protein present on Coomassie blue—stained polyacrylamide gels after electrophoresis of bacterial cells and culture filtrates.

In addition to the experiments described above, we investigated the effect on glutamine synthetase expression of the antisense PS-ODN #269-275 with a single nucleotide mismatch at codon 270 (third nucleotide position) of the glutamine synthetase target site. This PS-ODN, mismatched at 1 nucleotide, inhibited glutamine synthetase expression to the same extent as the perfectly matched antisense PS-ODN. This PS-ODN also inhibited M. tuberculosis growth to the same extent as the perfectly matched antisense PS-ODN.

### Example 5

Effect of PS-ODNs on the Amount of Cell Wall poly-L-glutamate/glutamine.

Bacterial cultures, inoculated at 1 or 5×10⁵ cells/ml and grown for 6 weeks (M. tuberculosis) or 6 days (M. smegmatis), were analyzed for the amount of poly-L-glutamate/glutamine and D,L-alanine in the cell wall as described (Wietzerbin et al., (1975) Biochem. Biophys. Res. Comm. 62, 246-252). Briefly, disrupted cells were treated with trypsin and chymotrypsin, delipidated by acetone and chloroform/methanol extractions, and acid hydrolyzed. The redissolved hydrochloric acid precipitate contained the poly-L-glutamate/glutamine fraction, while the insoluble portion contained the peptidoglycan chain. Both fractions were analyzed for their amino acid content after complete acid hydrolysis.

Determination of the Amount of poly-L-glutamate/glutamine in the Cell Wall of M. tuberculosis.

Pathogenic, but not nonpathogenic mycobacteria, release abundant amounts of glutamine synthetase and contain a large amount of poly-L-glutamate/glutamine in the bacterial cell wall. The high correlation between glutamine synthetase export and the presence of poly-L-glutamate/glutamine in the bacterial cell wall suggests the possibility that extracellular glutamine synthetase is involved in the synthesis of this cell wall structure. If so, then inhibition of extracellular glutamine synthetase might reduce the amount of this structure. We investigated this hypothesis by determining the amount of this cell wall structure in M. tuberculosis grown in the presence of various antisense and control PS-ODNs at their effective concentration of 10 μM for 6 weeks by which time the bacteria had reached stationary phase. The heteropolymer was isolated from the mycobacterial cell wall to a purity of 90-95% and, along with the co-purified mycobacterial peptidoglycan moiety, subjected to total amino acid hydrolysis (Table 2).

PS-ODNs #4-9 or #269-275, separately or in combination, reduced the amount of poly-L-glutamate/glutamine from 50.3 μg pet 1×10¹⁰ cells in untreated control cultures to 46.7 μg in the presence of PS-ODN #4-9, to 43.5 μg in the presence of PS-ODN #269-275, and to 38.3 μg in the presence of both PS-ODNs. The combination of all control PS-ODNs together did not significantly decrease the amount of this structure (Table 2). Quantitatively, the decrease in poly-L-glutamate/glutamine (23.8±3.1%) compares favorably with the decrease in detectable extracellular glutamine synthetase activity described above. Because the isolation procedure resulted in the hydrolysis of glutamine, we could not determine the exact ratio of glutamate:glutamine residues in the heteropolymer.

By comparison, PS-ODNs decreased the amount of D,L-alanine (present in equimolar amounts) in the peptidoglycan fraction (≧90% homogeneous) from 20.5 μg per 1×10¹⁰ cells in control cultures to 19.0 μg in the presence of PS-ODN #4-9, to 18.5 μg in the presence of PS-ODN #269-275, and to 17.4 μg in the presence of both PS-ODNs, a reduction of 15.1±0.3%. The combination of all control PS-ODNs did not decrease the amount of this structure. These results indicated that the inhibitory effect of the PS-ODNs was somewhat more pronounced on poly-L-glutamate/glutamine than on D,L-alanine.

### Example 6

Effect of PS-ODNs on the Growth of M. tuberculosis and M. smegmatis.

Growth of the cultures over 6 weeks (M. tuberculosis) or 6 days (M. smegmatis) was monitored weekly (M. tuberculosis) or daily (M. smegmatis) by gently sonicating the cultures to break up clumped bacteria, removing a small aliquot, washing the bacteria, plating serial dilutions of the washed bacteria on 7H11 agar (Difco), and enumerating colony forming units after incubation for 2 weeks (M. tuberculosis ) or 3 days (M. smegmatis). Standard deviations are given in the relevant figure legends.

Effect of Antisense PS-ODNs on M. tuberculosis and M. smegmatis Proliferation.

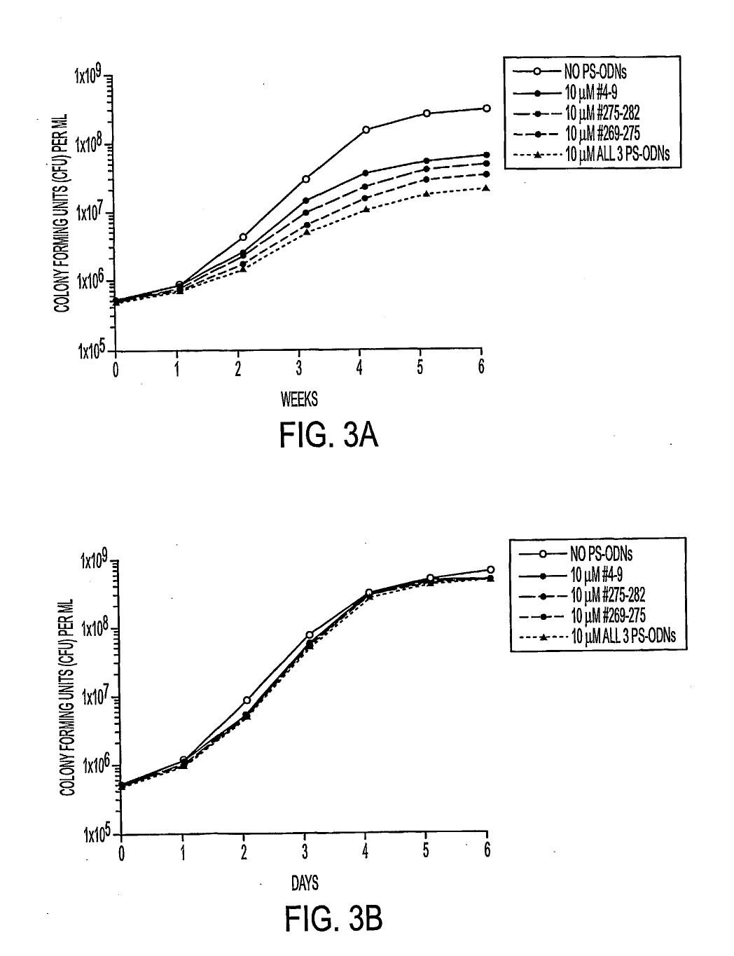

In the course of evaluating the cellular and extracellular glutamine synthetase activity of the two mycobacterial species cultured in the presence of various combinations of antisense PS-ODNs at concentrations of 10 μM, we noticed a reduction in cell density in M. tuberculosis but not M. smegmatis cultures after their standard growth period. We therefore explored the capacity of antisense PS-ODNs to inhibit bacterial growth. We added PS-ODNs to cultures of M. tuberculosis Erdman and cultures of both the patent and recombinant strain of M. smegmatis 1-2c and quantified colony forming units weekly for 6 weeks (M. tuberculosis) or daily for 6 days (M. smegmatis) (FIG. 3).

All three antisense PS-ODNs exerted a substantial inhibitory effect on the growth of M. tuberculosis, causing inhibited cultures to lag 0.7-0.9 log units (±0.1-0.2 log units; S.D.) behind uninhibited cultures at the end of the growth period (FIG. 3A). The magnitude of an individual PS-ODN's capacity to inhibit growth paralleled its capacity to inhibit M. tuberculosis glutamine synthetase activity: PS-ODN #269-275>#275-282>#4-9. Combinations of 2 PS-ODNs had a slightly greater inhibitory effect on the cultures than single PS-ODNs, decreasing growth by up to 1.0 log units, and the combination of all three PS-ODNs decreased growth by 1.25 log units (±0.2 log units; S.D.). Interestingly, in contrast to the results with glutamine synthetase activity, at a concentration of 1 μM, there was a small but measurable growth inhibitory effect of the PS-ODNs, 0.1 to 0.25 log units. There was no evidence that any of the M. tuberculosis cultures were able to escape the effect of the PS-ODNs, i.e. develop resistance to the antisense PS-ODNs. At all concentrations and combinations tested, the control PS-ODNs did not affect M. tuberculosis multiplication.

None of the antisense or control PS-ODNs (mismatched at 2-4 nucleotide positions with the endogenous M. smegmatis enzyme), in the range of 0.1-10 μM, in any possible combination, either in the free form or tethered to an amikacin moiety, had any growth inhibitory effect on either strain of M. smegmatis (FIG. 3B).

### Example 7

Effect of Combinations of Anti-Tuberculosis Drugs and Glutamine Synthetase Antisense ODNs on M. tuberculosis and M. smegmatis Proliferation.

Several anti-tuberculosis drugs, such as isoniazid or ethambutol, affect the membrane metabolism of M. tuberculosis (see e.g. Mdluli et al., (1998) Science 280, 1607-1610 and Telenti et al., (1997) Nat. Med. 3, 567-570). At high drug concentrations, the damage in the mycobacterial cell wall becomes so extensive that the bacteria can no longer control influx and efflux of ions, proteins, and other vital cellular components, and the bacteria subsequently lyse. We reasoned that subinhibitory concentrations of such drugs right “soften” the cell wall and promote the influx of PS-ODNs, thereby enhancing their effectiveness. Two drugs, ethambutol and polymyxin B nonapeptide, were chosen for this purpose.

We first determined the minimal inhibitory concentrations (MIC) of ethatnbutol and polymyxin B nonapeptide, for M. tuberculosis Erdman and M. smegmatis 1-2c. The MIC of ethambutol was ˜5 μg/ml for M. tuberculosis Erdman and ˜10 ng/ml for M. smegatis 1-2c and the MIC of polymyxin B nonapeptide was ˜5 μg/ml for both M. tubercilosis Erdman and M. smegmatis 1-2c. The MICs for the parent and recombinant strains of M. smegmatis 1-2c were identical

We then incubated the 3 strains of bacteria with subinhibitory concentrations of 0, 0.1, 0.25, and 0.5 μg/ml of ethambutol, polymyxin B nonapeptide, or both antibiotics and with or without each PS-ODN alone or all 3 PS-ODNs in combination.

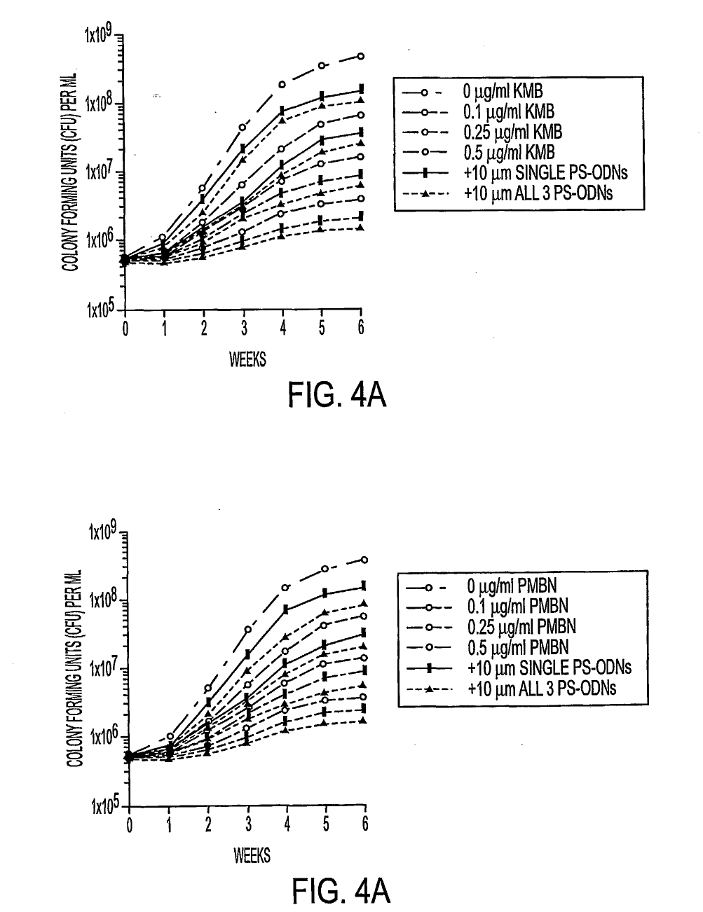

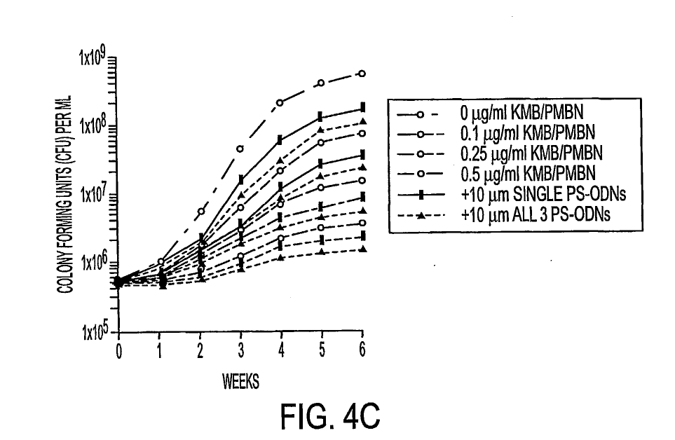

The addition of antisense PS-ODNs to M. tuberculosis cultures containing subinhibitory concentrations of one or both antibiotics resulted in additional growth inhibition beyond that caused by the antibiotic alone (FIG. 4). However, the incremental increase in growth inhibition was not greater than the amount of growth inhibition observed in cultures treated with PS-ODNs in the absence of antibiotics. Indeed, with increasing concentrations of antibiotic, the magnitude of the incremental increase in growth inhibition caused by the PS-ODNs diminished.

None of the antisense or control PS-ODNs, in the range of 0.1-10 μM, in any possible combination, either in their free form or tethered to an amikacin moiety, enhanced the growth inhibitory effects of ethambutol and/or polymyxin B nonapeptide on either strain of M. smegmatis.

### Example 8

PS-ODNs Directed Against the aroA, ask and groES Genes of M. tuberculosis

PS-ODNs:

The following PS-ODNs against the aroA gene were tested, all at 10 μM:

Val 23:5′-TGGGGCTGGCCATGTCTTCAC-3′ (SEQ ID NO: 7) 21-met fully complementary to nucleotides 1-21 of the aroA gene starting from the start codon. Strong propensity to remain linear.

Val 24:5′-CGCTTCATCCTGCCGTGTCGG (SEQ ID NO: 8) 21-mer fully complementary to nucleotides −21 to −1 of the upstream non-coding sequence of the aroA gene. Moderate propensity to remain linear.

Val 25:5′-TGGCCATGTCTTCACCGCTTCATCCTG (SEQ ID NO: 9) 27-mer fully complementary to nucleotides −12 to 15 of the aroA gene. Strong propensity to remain linear.

Val 26:5′-CCATGTCTTCACCGCTTCATCCTG (SEQ ID NO: 10) 24-mer fully complementary to nucleotides −12 to 12 of the aroA gene. Moderate propensity to remain linear.

The following PS-ODNs against the M. tuberculosis ask gene were tested, both at 10 μM:

Val 33 (aka ODS 33): 5′-TGCCGCAGCCACGGCGACGGCCGTGGT (SEQ ID NO: 11) 27-met complementary to the nucleotide sequence 466-492 of the ask gene Mismatched at 5 nucleotides, but 2 involve potentially stable C:C base pairs) (This 27-mer is fully complementary to the nucleotide sequence 1527-1553 of the M. smegmatis ask-asd operon).

Val 34 (aka ODS 55): 5′-TGCCGCAGCCACGGCGACGGCCGTGGTGTCCGAACC (SEQ ID NO: 12) 36-met complementary to the nucleotide sequence 457-492 of the ask gene (Mismatched at 6 nucleotides, but 2 involve potentially stable C:C base pairs) (This 36-mer is fully complementary to the nucleotide sequence 1518-1553 of the M smegmatis ask-asd operon).

The following PS-ODN against the groES (10 kDa antigen) gene was tested at 10 μm:

Val 41: 5′-CACCTTCGCCACGAITGGAGCCCTCCA (SEQ ID NO: 13) 27-mer complementary to the nucleotide sequence −15 to 12 of the groES gene.

Methods:

M. tuberculosis Erdman strain was grown in 7H9 medium to an Optical Density (O.D.) at 540 nm of 0.5, sonicated, diluted in 7H9 medium to an O.D. of approximately 0.05, and 2 ml of the suspension was added to replicate 12×75 mm plastic test tubes. A single PS-ODN (i.e. one of the above 7 PS-ODNs [SEQ ID NOs: 7-13]) at a final concentration of 10 μM or control buffer was added to each tube. In addition, one complete set of 8 tubes (7 tubes with one of the PS-ODNs and one tube with control buffer) was incubated with ethambutol at a final concentration of 5 μg/mL The cultures were incubated for 6 weeks. Growth of M. tuberculosis was assayed by obtaining O.D. measurements weekly.

Results:

Val 23 inhibited M. tuberculosis growth in the absence of antibiotic. It enhanced the inhibition of M. tuberculosis in the presence of antibiotic.

Val 24 did not inhibit M. tuberculosis growth in the absence of antibiotic. It enhanced the inhibition of M. tuberculosis in the presence of antibiotic.

Val 25 did not inhibit M. tuberculosis growth in the absence of antibiotic nor enhance the inhibition of M. tuberculosis in the presence of antibiotics.

Val 26 did not inhibit M. tuberculosis growth in the absence of antibiotic. It enhanced the inhibition of M. tuberculosis in the presence of antibiotic.

Val 33 inhibited M. tuberculosis growth in the absence of antibiotic. It enhanced the inhibition of M. tuberculosis in the presence of antibiotic.

Val 34 inhibited M. tuberculosis growth in the absence of antibiotic. It enhanced the inhibition of M. tuberculosis in the presence of antibiotic.

Val 41 did not inhibit M. tuberculosis growth in the absence of antibiotic. It enhanced the inhibition of M. tuberculosis in the presence of antibiotic.

Graphs:

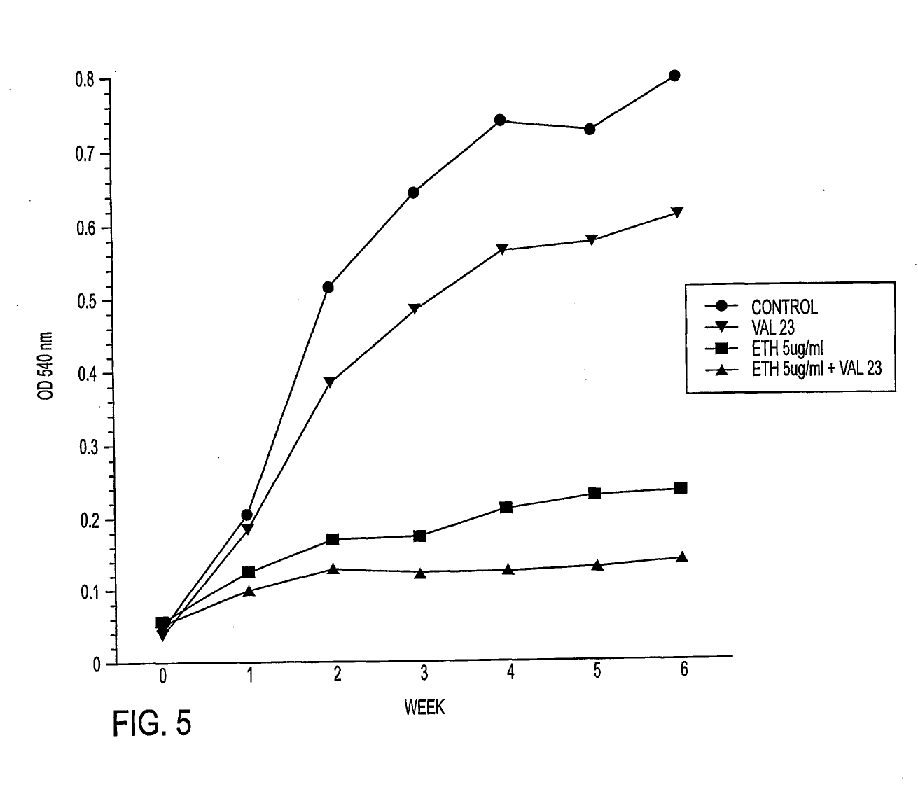

FIG. 5 shows a graph of Val 23 with and without Ethambutol (ETH) (5 μg/ml).

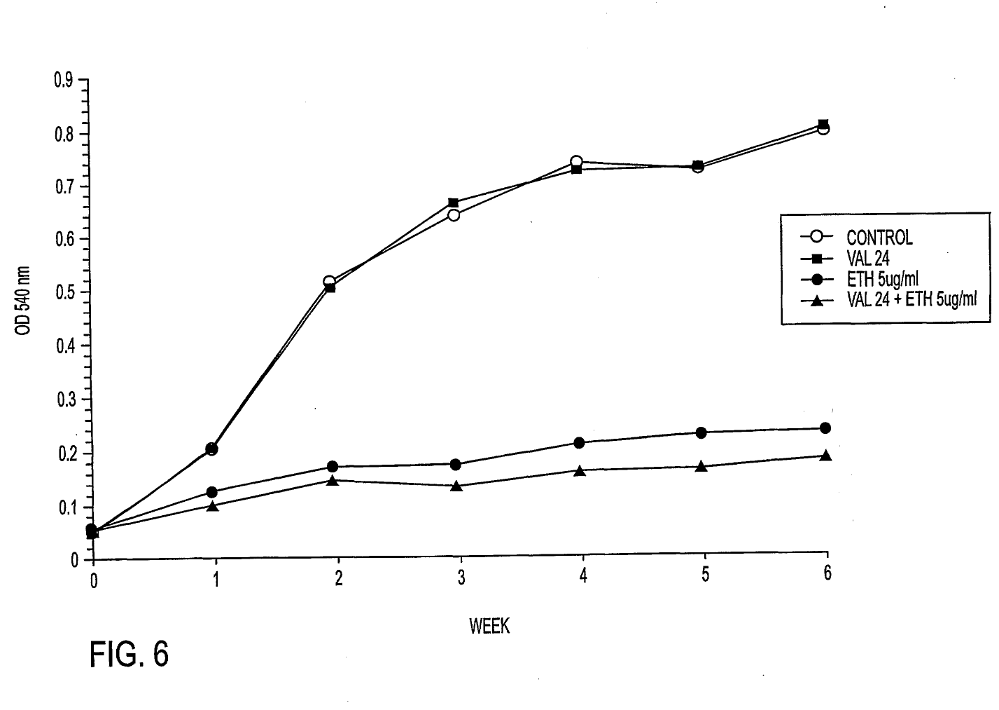

FIG. 6 shows a graph of Val 24 with and without Ethambutol (ETH) (5 μg/ml).

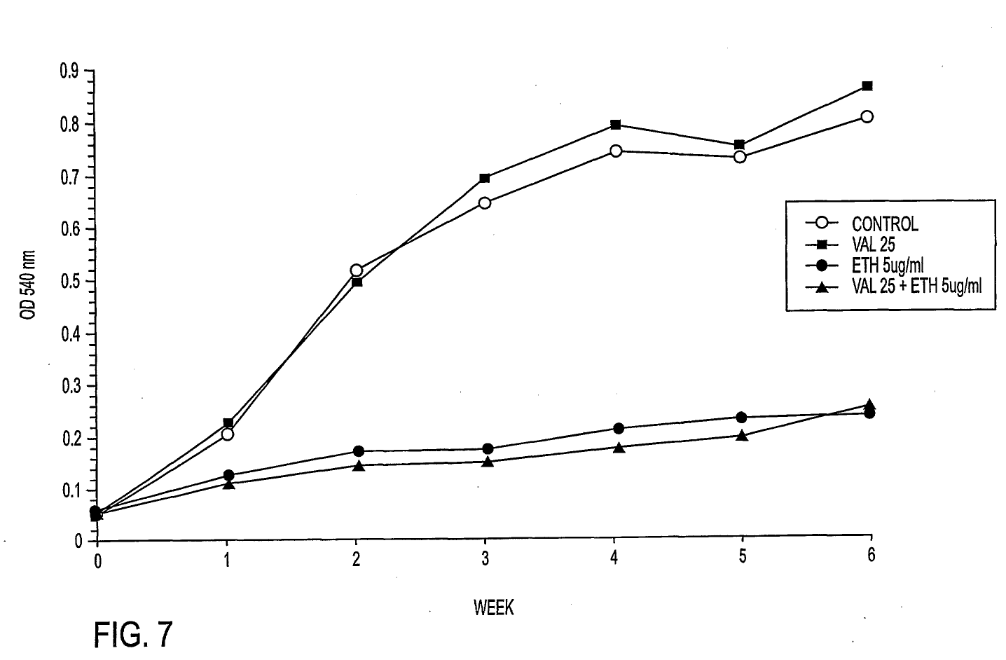

FIG. 7 shows a graph of Val 25 with and without Ethambutol (ETH) (5 μg/ml).

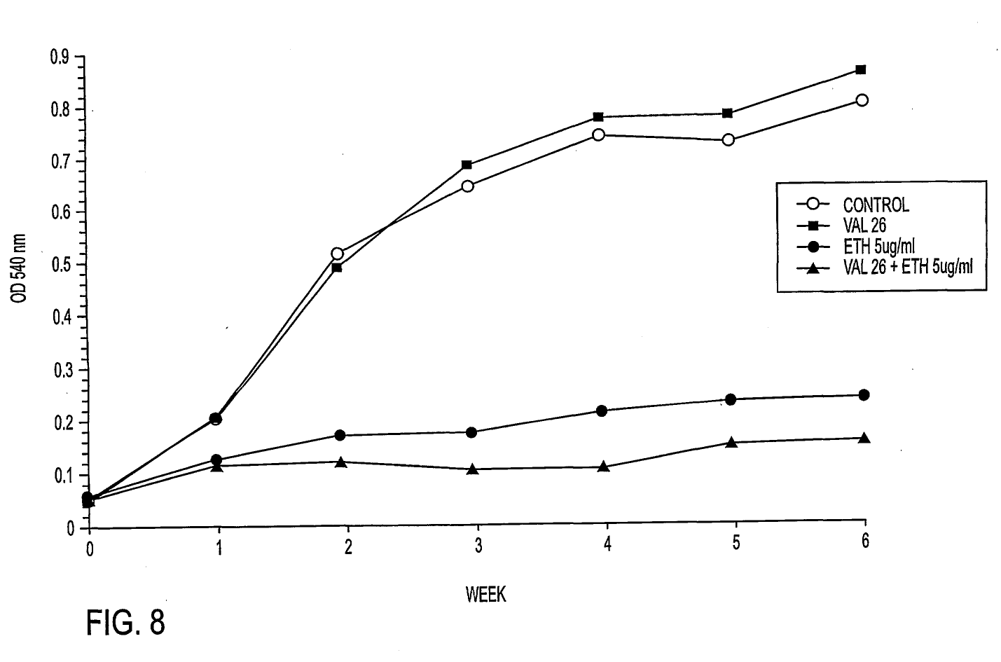

FIG. 8 shows a graph of Val 26 with and without Ethambutol (ETH) (5 μg/ml).

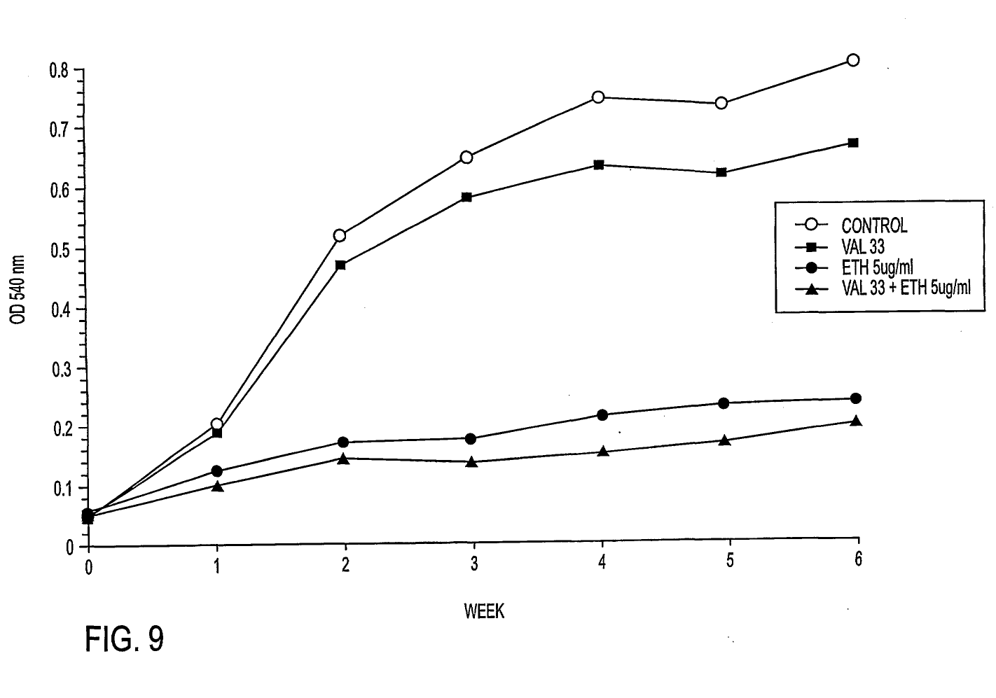

FIG. 9 shows a graph of Val 33 with and without Ethambutol (ETH) (5 μg/ml).

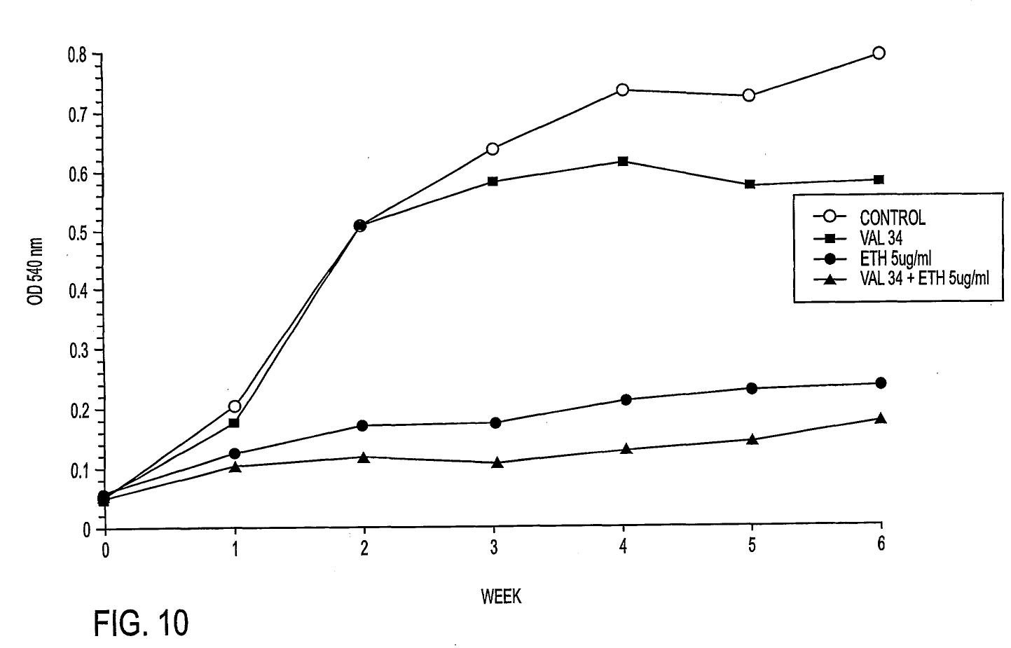

FIG. 10 shows a graph of Val 34 with and without Ethambutol (ETH) (5 μg/ml)

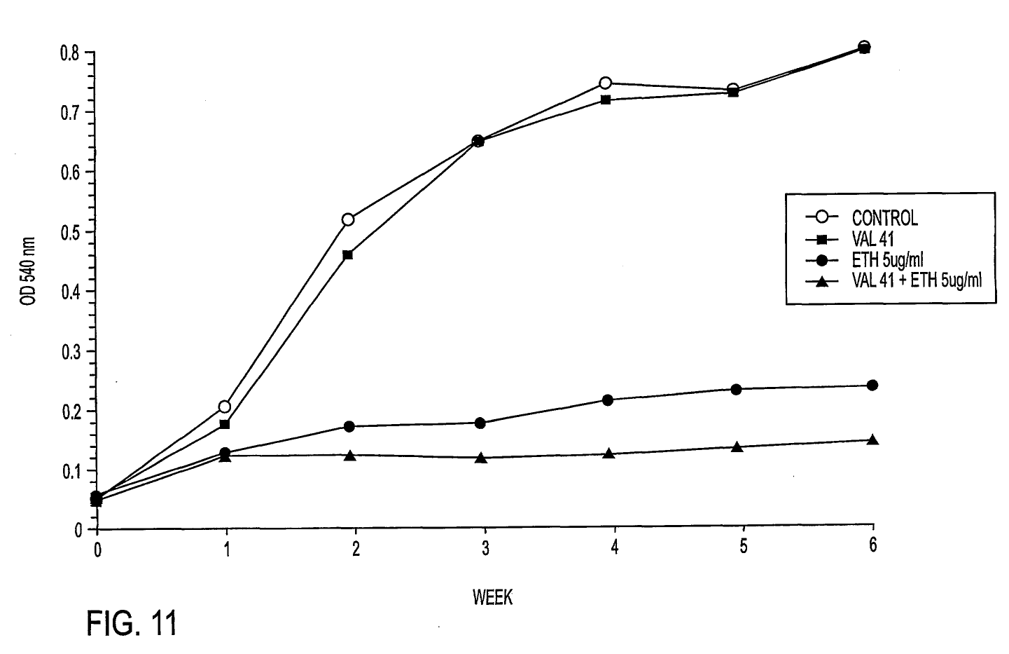

FIG. 11 shows a graph of Val 41 with and without Ethambutol (ETH) (5 μg/ml).

### Example 9

PS-ODNs Directed Against the 30/32 kDa Protein Complex Genes of M. tuberculosis

PS-ODNs:

The following PS-ODN against the 30/32 kDa protein complex genes was tested at 10 μM:

ML10: 5-GAACGCCGGGGTGTTGATGTCCCAGCCG (SEQ ID NO: 14) 28-mer complementary to: a) nucleotides 276-303 of the 32A kDa protein gene from the entire coding region including leader sequence)(No mismatches); b) nucleotides 267-294 of the 30 kDa protein gene (Mismatched at position 276); c) nucleotides 279-306 of the 32 kDa protein gene (Mismatched at position 303).

Methods:

M. tuberculosis Erdman strain was grown in 7H9 medium to an Optical Density (O.D.) at 540 nm of 0.5, sonicated, diluted in 7H9 medium to an O.D. of approximately 0.05, and 2 ml of the suspension was added to replicate 12×75 mm plastic test tubes. Three set of tubes were prepared. Set 1: The PS-ODN described above at a final concentration of 10 μM or control buffer was added to each tube in duplicate once only at the start of the experiment Set 2: The PS-ODN described above at a final concentration of 10 μM or control buffer was added to each tube in duplicate weekly for 9 weeks. Set 3: The PS-ODN described above at a final concentration of 10 μM or control buffer was added to each tube in duplicate once only at the start of the experiment and the tubes were additionally incubated with ethambutol at a final concentration of 5 μg/mL The cultures were incubated for 9 weeks. Growth of M. tuberculosis was assayed by obtaining O.D. measurements weekly.

Results:

ML-10 added only once at the start of the experiment slightly inhibited M. tuberculosis growth relative to control (Buffer only) in the absence of antibiotic.

ML-10 added weekly for 9 weeks inhibited M. tuberculosis growth relative to control (Buffer only added weekly for 9 weeks) in the absence of antibiotic.

ML-10 added only once at the start of the experiment slightly enhanced the inhibition of M. tuberculosis growth in the presence of the antibiotic ethambutol.

Graph:

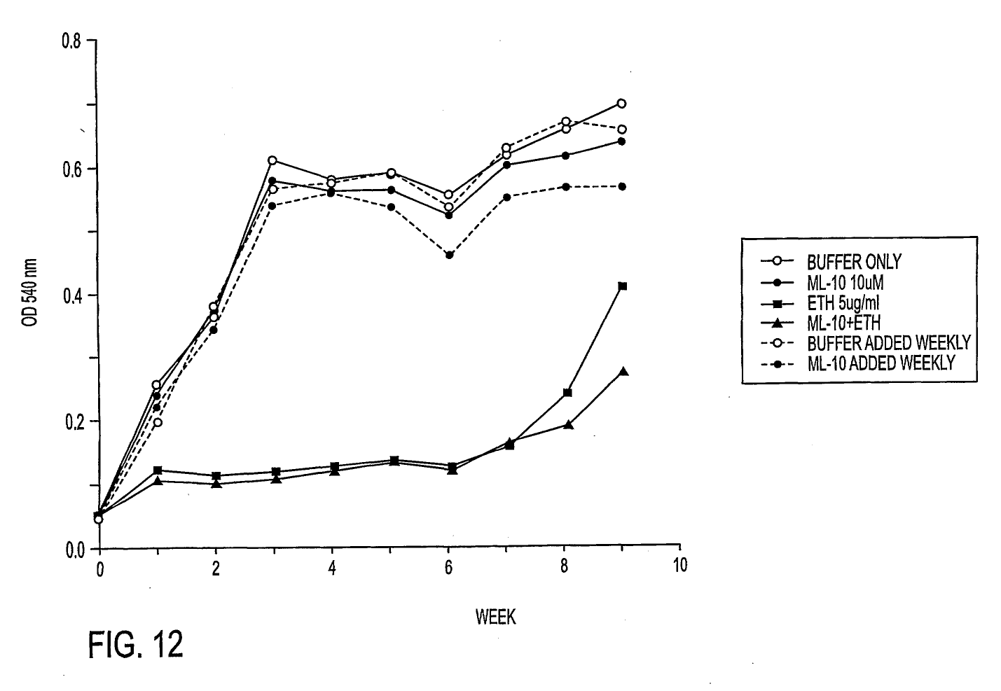

FIG. 12 shows a graph of ML10 with and without Ethatnbutol (ETH) (5 μg/ml) and added once vs. weekly.

### Example 10

Experiments with Modified PS-ODNs Against the Glutamine Synthetase Gene (modifications of 269-275)

Experiment I:

PS-ODNs: The following PS-ODNs were tested, all at 10 μM:

1. 269-275-DAO. PS-ODN 269-275 modified with a diaminooctane group at the base next to the 3′-terminal. All of the other internucleoside residues are P-S. 5′-C ATG CCG GAC CCG TTG TCG C*C-3′ (SEQ ID NO 19)*=DAO

2. 269-275 PS-ODN 269-275.

3. Control Nonsense ODN for 269-275 as described in Harth et al., PNAS 97(1): 418-423 (2000) [which is incorporated herein by reference] (middle base of each codon mismatched).

Methods:

M. tuberculosis Erdman strain was grown in 7H9 medium to an Optical Density (O.D.) at 540 nm of 0.5, sonicated, diluted in 7H9 medium to an O.D. of approximately 0.05, and 2 ml of the suspension was added to replicate 12×75 mm plastic test tubes. A single PS-ODN among the three listed above at a final concentration of 10 μM or control buffer was added to each tube. The cultures were incubated for 6 weeks. Growth of M. tuberculosis was assayed by obtaining O.D. measurements weekly and enumerating colony forming units on 7H11 agar medium.

Results:

1. 269-275-DAO was more effective at 10 μM than PS-ODN 269-275 at inhibiting the growth of M. tuberculosis. The control nonsense ODN yielded no inhibition of growth, i.e. it was equivalent to no ODN being added.

2. 269-275-DAO did not inhibit M. smegmatis, as expected.

Graphs:

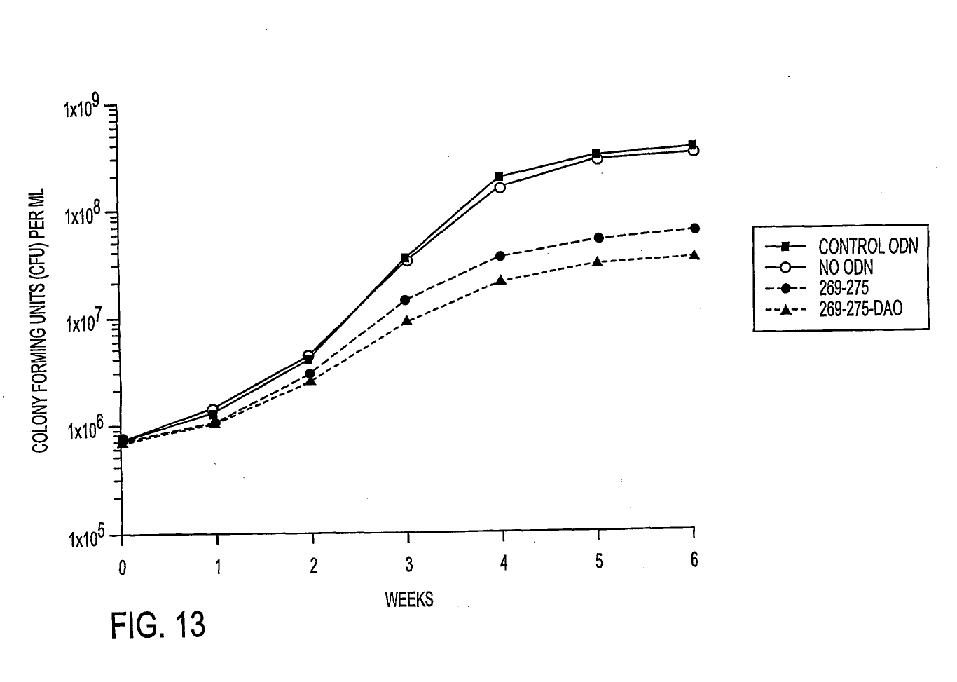

FIG. 13 shows that 269-275-DAO was mote effective at 10μM than PS-ODN 269-275 at inhibiting the growth of M. tuberculosis. The control ODN yielded no inhibition of growth, i.e. it was equivalent to no ODN being added.

FIG. 14 shows that 269-275-DAO does not inhibit M. smegmatis.

Experiment II:

PS-ODNs: The following PS-ODNs were tested, all at 10 μM:

1. 1 N-269-275. 1 amino group covalently attached to the 5′ terminus.

5′-1 N-CAT GCC GGA CCC GTT GTC GCC-3′ (SEQ ID NO 19)

2. 2N-269-275. 2 amino groups covalently attached between the second to last and last base of the 3′ terminus.

5′-CAT GCC GGA CCC GTT GTC GCC-2N-G-3′ (SEQ ID NO 19)

3. 3N-269-275. 3 amino groups covalently attached to the 5′ terminus.

5′-3N-CAT GCC GGA CCC GTT GTC GCC-3′ (SEQ ID NO 19)

4. 4N-269-275. 2 amino groups covalently attached to the 5′ terminus and 2 amino groups covalently attached between the second to last and last base of the 3′ terminus.

5′-2N-CAT GCC GGA CCC GTT GTC GCC-2N-G-3′ (SEQ ID NO 19)

5. 5N-269-275. 2 amino groups covalently attached to the 5′ terminus and 3 amino groups covalently attached between the second to last and last base of the 3′ terminus.

5′-2N-CAT GCC GGA CCC GTT GTC GCC-3N-G-3′ (SEQ ID NO 19)

6. 269-275=PS-ODN 269-275.

Methods:

M. tuberculosis Erdman strain was grown in 7H9 medium to an Optical Density (O.D.) at 540 nm of 0.5, sonicated, diluted in 7H9 medium to an O.D. of approximately 0.05, and 2 ml of the suspension was added to replicate 12×75 mm plastic test tubes. A single PS-ODN among the six listed above at a final concentration of 10 μM or control buffer was added to each tube. The cultures were incubated for 6 weeks. Growth of M. tuberculosis was assayed by obtaining O.D. measurements weekly and enumerating colony forming units on 7H11 agar medium.

Results:

Only the 5N-269-275 PS-ODN was more effective than PS-ODN 269-275. Perhaps more importantly, there was a trend toward greater inhibition with higher “N” groups. (5N>4N>3N/2N/1N).

Graphs:

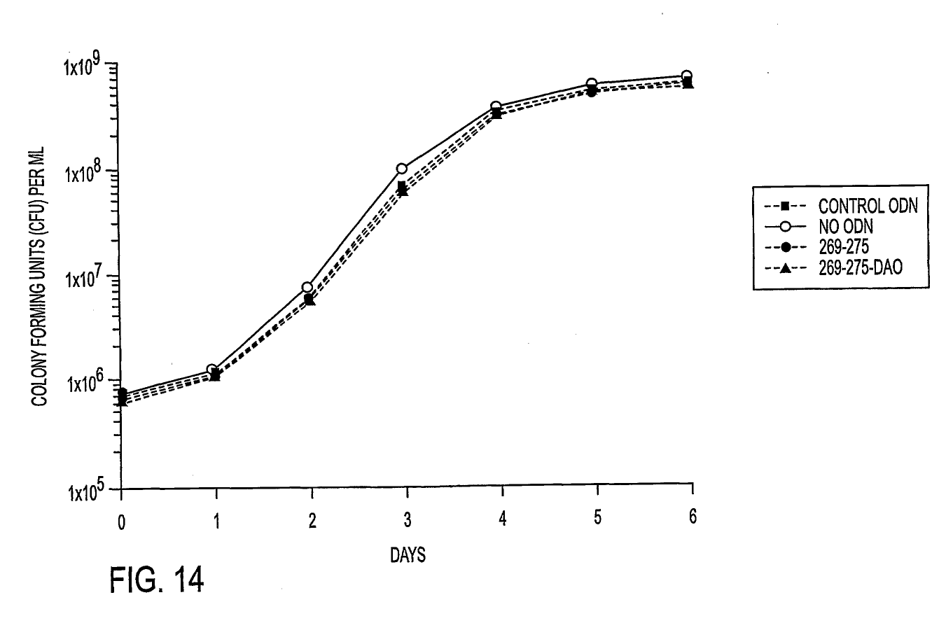

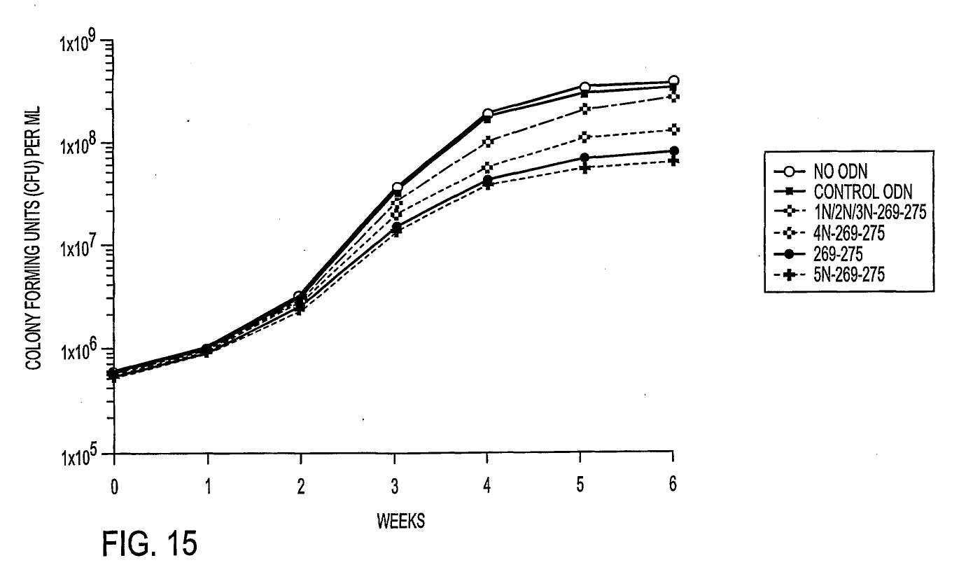

FIG. 15 shows that only 5N-269-275 PS-ODN was more effective than PS-ODN 269-275 and that there was a trend toward greater inhibition with higher “N” groups. (5N>4N>3N/2N/1N).

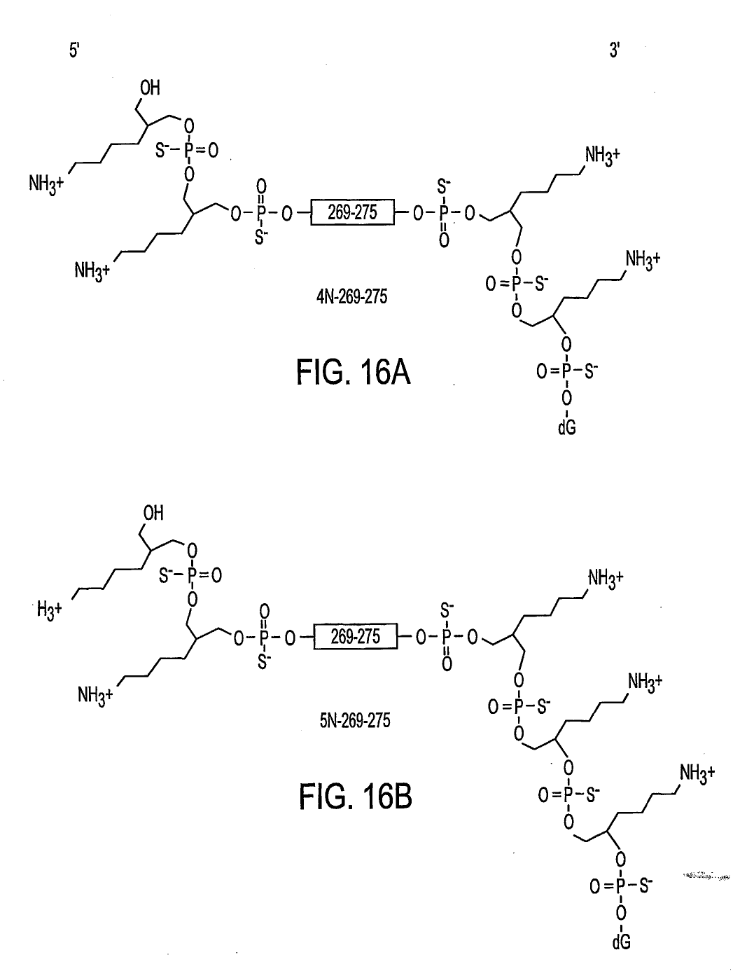

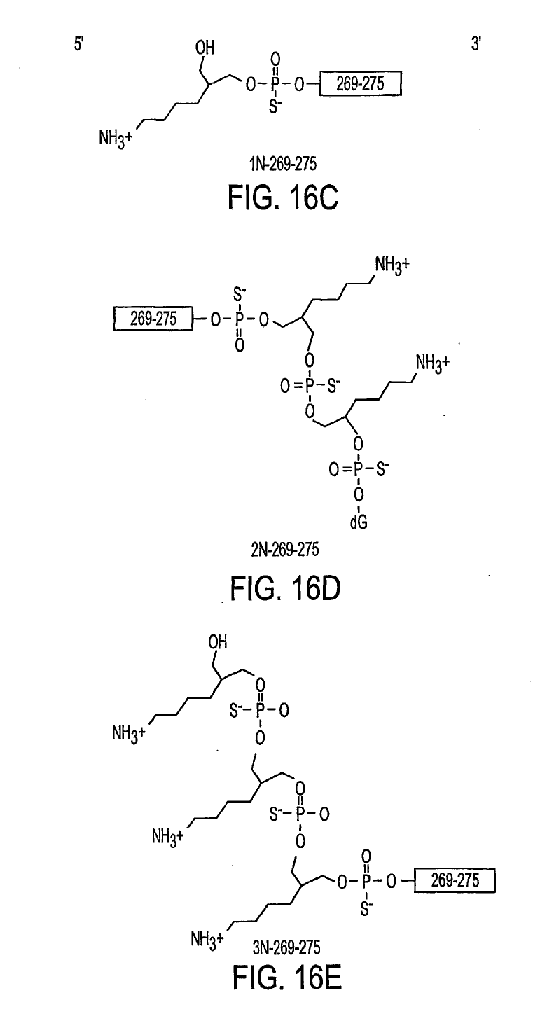

FIG. 16 shows the six ODNs tested in this experiment.

TABLE I
TARGET SITES OF ANTISENSE PS ODNs SPECIFIC FOR M. TUBERCULOSIS ERDMAN GLUTAMINE SYNTHETASE mRNA

| | ANTISENSE PS-ODN | ANTISENSE PS-ODN | ANTISENSE PS-ODN |
|---|---|---|---|
| | #4-9 | #269-275 | #275-282 |
| glnA1 | 5′-AAG ACG CCC GAC GAC GTC | 5′-GGC GAC AAC GGG TCC GGC ATG | 5′-ATG CAC TGT CAT CAG TCG CTG TGG |
| CODING | | | |
| REGION | | | |
| glnA1 | 5′-AAG ACG CCC GAC GAG GUC | 5′-GGC GAC AAC GGG UCC GGC AUG | 5′-AUG CAC UGU CAU CAG UCG CUG UGG |
| TRANSCRIPT | | | |
| PS-ODN | 5′-GAC GTC GTC GGG CGT CTT | 5′-CAT GCC GGA CCC GTT GTC GCC | 5′CCA CAG CGA CTG ATG ACA GTG CAT |
| SEQUENCE | | | |
| PS-ODN | 18 | 21 | 24 |
| LENGTH [nt] | | | |
| PS-ODN | 66.7 | 71.4 | 54.2 |
| GC CONTENT | | | |
| [%] | | | |
| PROPENSITY | WEAK | STRONG | MODERATE |
| OF PS-ODN | | | |
| TO REMAIN | | | |
| LINEAR | | | |

Abbreviations used:
PS-ODN, phosphorothioate modified oligodeoxyribonucleotide;
glnA1, glutamine synthetase gene;
[nt], nucleotide(s)

TABLE II
EFFECT OF VARIOUS ANTISENSE PS-ODNs SPECIFIC FOR
M. TUBERCULOSIS ERDMAN GLUTAMINE SYNTHETASE
mRNA THE CELL WALL COMPONENT POLY-L-GLUTAMATE/
GLUTAMINE AND THE PEPTIDOGLYCAN CHAIN

| | M. TUBERCULOSIS | |
|---|---|---|
| | ERDMAN POLY-L- | M. TUBERCULOSIS |
| | GLUTAMATE/ | ERDMAN D, L- |
| | GLUTAMINE | ALANINE |
| | [μg/1 × 10¹⁰ | [μg/1 × 10¹⁰ |
| | BACTERIA] | BACTERIA] |
| WILD TYPE | 50.3 +/− 0.6^{b} | 20.6 +/− 0.4 |
| PS-ODN #4-9^{a} | 46.7 +/− 0.6 | 19.0 +/− 0.6 |
| PS-ODN #269-275 | 43.5 +/− 1.6 | 18.5 +/− 0.4 |
| PS-ODNs #4-9 + | 38.3 +/− 1.1 | 17.5 +/− 0.4 |
| #269-275 | | |
| CONTROL PS-ODNs | 49.5 +/− 0.9 | 20.4 +/− 0.5 |
| #4-9 + #269-275 + | | |
| #275-282 | | |

^{a}All PS-ODNs used at 10 μM
^{b}Mean +/− S.D. for two independent analyses

1. A method of inhibiting Mycobacterium tuberculosis glutamine synthetase protein expression comprising contacting a Mycobacterium tuberculosis bacterium with an effective amount of an antisense compound comprising an antisense polynucleotide that hybridizes to a Mycobacterium tuberculosis glutamine synthetase polynucleotide, wherein the antisense polynucleotide hybridizes to a region of the Mycobacterium tuberculosis glutamine synthetase polynucleotide encoding the glutamine synthetase protein, thereby inhibiting Mycobacterium tuberculosis glutamine synthetase protein expression.

2. The method of claim 1, wherein the polynucleotide has modification to its internucleoside phosphates linkages selected from the group consisting of phosphorothioates, methylphosphonates, phosphoroboronates, phosphoromorpholidates, butyl amidates, and peptide nucleic add linkages.

3. The method of claim 2, wherein the polynucleotide is selected from the group consisting of 5′-GAC GTC GTC GGG CGT CTT-3′ (SEQ ID NO: 18), 5′-CAT GCC GGA CCC GTT GTC GCC-3′ (SEQ ID NO: 19) and 5′-CCA CAG CGA CTG ATG ACA GTG CAT-3′ (SEQ ID NO: 20).

4. The method of claim 1, further comprising contacting the Mycobacterium tuberculosis with an effective amount of an antibiotic capable of inhibiting the proliferation of Mycobacterium tuberculosis.

5. The method of claim 4, wherein the antibiotic is selected from the group consisting of rifampin, isoniazid, amikacin, ethambutol and polymyxin B nonapeptide.

6. The method of claim 1, further comprising contacting the Mycobacterium tuberculosis with an effective amount of a second antisense polynucleotide that hybridizes to a Mycobacterium tuberculosis glutamine synthetase polynucleotide, wherein the second antisense polynucleotide hybridizes to a region of the Mycobacterium tuberculosis glutamine synthetase polynucleotide encoding the glutamine synthetase protein that is distinct from the region targeted by the antisense polynucleotide of claim 1.

7. The method of claim 1, further comprising contacting the Mycobacterium tuberculosis with an effective amount of a second antisense polynucleotide that hybridizes to a Mycobacterium tuberculosis polynucleotide selected from the group consisting of polynucleotides that encode AroA, Ask, GroES or the 30, 32A, 32B extracellular proteins.

8. The method of claim 1, wherein the antisense polynucleotide inhibits the growth of Mycobacterium tuberculosis.

9. An antisense compound of about 15 to about 50 nucleobases in length targeted to a portion of a nucleic acid molecule encoding a Mycobacterium tuberculosis glutamine synthetase protein, wherein the antisense compound specifically hybridizes with the nucleic acid molecule encoding the Mycobacterium tuberculosis glutamine synthetase protein, thereby inhibiting the expression of the Mycobacterium tuberculosis glutamine synthetase protein.

10. The antisense compound of claim 9 which is an antisense oligonucleotide.

11. The antisense compound of claim 9, wherein the antisense polynucleotide comprises 5′-GAC GTC GTC GGG CGT CTT-3′ (SEQ ID NO: 18), 5′-CAT GCC GGA CCC GTT GTC GCC-3′ (SEQ ID NO: 19) or 5′-CCA CAG CGA CTG ATG ACA GTG CAT-3′ (SEQ ID NO: 20).

12. The antisense compound of claim 9 which comprises at least one modified internucleoside linkage.

13. The antisense compound of claim 12, wherein the modified internucleoside linkage is a phosphorothioate linkage.

14. The antisense compound of claim 9, wherein the antisense polynucleotide has a complete homology with the nucleic acid molecule encoding the Mycobacterium tuberculosis glutamine synthetase protein.

15. A process for producing an antisense compound that inhibits the expression of Mycobacterium tuberculosis glutamine synthetase comprising the steps of:
(a) synthesizing a antisense polynucleotide of about 15 to about 50 nucleobases in length, wherein the antisense polynucleotide is capable of hybridizing to a portion of a Mycobacterium tuberculosis polynucleotide encoding a Mycobacterium tuberculosis glutamine synthetase protein;
(b) contacting a culture of Mycobacterium tuberculosis with an effective amount of the antisense polynucleotide of step (a) such that the antisense polynucleotide hybridizes with a Mycobacterium tuberculosis glutamine synthetase polynucleotide;
(c) comparing the levels of Mycobacterium tuberculosis glutamine synthetase protein expression in the culture contacted with the effective amount of the antisense polynucleotide to the levels of Mycobacterium tuberculosis glutamine synthetase protein expression in a control culture of Mycobacterium tuberculosis, wherein the control culture is not contacted with the antisense polynucleotide; and
(d) determining whether the antisense polynucleotide inhibits the expression of glutamine synthetase by observing the level of glutamine synthetase expression in the Mycobacterium tuberculosis culture contacted with the antisense polynucleotide relative to the level of glutamine synthetase expression in the Mycobacterium tuberculosis control culture.

16. An antisense polynucleotide produced according to the process of claim 15.

17. A method of inhibiting protein expression of a Mycobacterium tuberculosis gene selected from the group consisting of AroA, Ask, GroES or the 30, 32A, 32B extracellular proteins comprising contacting Mycobacterium tuberculosis with an effective amount of an antisense polynucleotide that hybridizes to a Mycobacterium tuberculosis AroA, Ask, GroES, 30, 32A or 32B extracellular protein polynucleotide, wherein the antisense polynucleotide hybridizes to a region of the Mycobacterium tuberculosis polynucleotide encoding the protein.

18. An antisense compound of about 15 to about 50 nucleobases in length targeted to a portion of a nucleic acid molecule encoding a Mycobacterium tuberculosis protein selected from the group consisting of AroA, Ask, GroES, 30, 32A and 32B extracellular proteins, wherein the antisense compound specifically hybridizes with nucleic acid molecule encoding a Mycobacterium tuberculosis AroA, Ask, GroES, 30, 32A or 32B extracellular protein, thereby inhibiting the expression of the Mycobacterium tuberculosis AroA, Ask, GroES, 30, 32A or 32B extracellular protein.

19. A process for producing an antisense compound that inhibits the expression of a Mycobacterium tuberculosis gene selected from the group consisting of AroA, Ask, GroES or 30, 32A, 32B extracellular proteins comprising the steps of.
(a) synthesizing a antisense polynucleotide of about 15 to about 50 nucleobases in length, wherein the antisense polynucleotide is capable of hybridizing to a portion of polynucleotide encoding a Mycobacterium tuberculosis AroA, Ask, GroES or 30, 32A, 32B extracellular proteins;
(b) contacting a culture of Mycobacterium tuberculosis with an effective amount of the antisense polynucleotide such that the antisense polynucleotides hybridizes to a Mycobacterium tuberculosis aroA, ask, groES or 30, 32A, 32B extracellular protein polynucleotide;
(c) comparing the levels of Mycobacterium tuberculosis AroA, Ask, GroES or 30, 32A, 32B extracellular protein expression in the culture contacted with the effective amount of the antisense polynucleotide to the levels of Mycobacterium tuberculosis AroA, Ask, GroES or 30, 32A, 32B extracellular protein expression in a control culture of Mycobacterium tuberculosis, wherein the control culture is not contacted with the antisense polynucleotide; and
(d) determining whether the antisense polynucleotide inhibits the expression of AroA, Ask, GroES or 30, 32A, 32B extracellular proteins by observing the level of glutamine synthetase expression in the Mycobacterium tuberculosis culture contacted with the effective amount of the antisense polynucleotide relative to the level of AroA, Ask, GroES or 30, 32A, 32B extracellular protein expression in the Mycobacterium tuberculosis control culture.

20. An antisense polynucleotide produced according to the process of claim 19.

21. A method of inhibiting Mycobacterium tuberculosis protein expression comprising contacting a Mycobacterium tuberculosis bacterium with an effective amount of an antisense compound comprising an antisense polynucleotide that hybridizes to a Mycobacterium tuberculosis polynucleotide, wherein the antisense polynucleotide hybridizes to a region of the Mycobacterium tuberculosis polynucleotide encoding a Mycobacterium tuberculosis protein, thereby inhibiting the expression of the protein.

22. The method of claim 21, wherein the protein is an extracellular protein.
